(19) United States
(12) Patent Application Publication
(10) Pub. No.: US 2024/0143838 A1
(43) Pub. Date: May 2, 2024

(54) APPARATUS AND A METHOD FOR ANONYMIZING USER DATA

(71) Applicant: Nference, Inc., Cambridge, MA (US)

(72) Inventors: Sankar Ardhanari, Chapel Hill, NC (US); Murali Aravamudan, Andover, MA (US)

(73) Assignee: Nference, Inc., Cambridge, MA (US)

(21) Appl. No.: 18/385,057

(22) Filed: Oct. 30, 2023

Related U.S. Application Data
(60) Provisional application No. 63/381,492, filed on Oct. 28, 2022.

(51) Int. Cl. G06F 21/62; G16H 10/60

(52) U.S. Cl. CPC G06F 21/6254; G16H 10/60

(57) ABSTRACT

An apparatus for anonymizing user data is disclosed. The apparatus includes at least a processor and a memory communicatively connected to the at least a processor. The memory instructs the processor to receive, from a first database, a plurality of user data comprising a plurality of metadata. The memory instructs the processor to detach the plurality of metadata for the plurality of user data. The memory instructs the processor to identify a plurality of patient identifiers within the plurality of user data and the plurality of metadata. The memory instructs the processor to generate a plurality of anonymized data and anonymized metadata as a function of the plurality of patient identifiers and the plurality of metadata using an anonymization machine learning model. The memory instructs the processor to construct a plurality of anonymized user records as a function of the plurality of anonymized data.

### CROSS-REFERENCE TO RELATED APPLICATIONS

This application claims the benefit of priority of U.S. Provisional Patent Application Ser. No. 63/381,492, filed on Oct. 28, 2022, and titled “SYSTEMS AND METHODS FOR PROCESSING MEDICAL DATA,” which is incorporated by reference herein in its entirety.

### FIELD OF THE INVENTION

The present invention generally relates to the field of data security. In particular, the present invention is directed to an apparatus and a method for anonymizing user data.

### BACKGROUND

Managing sensitive data has long been a labor intensive process. Data security threats have increased exponentially due to the escalating challenges of data security in an interconnected world. Current options to anonymize data have fallen short due to inaccuracies and time constraints.

### SUMMARY OF THE DISCLOSURE

In an aspect, an apparatus for anonymizing user data is disclosed. The apparatus includes at least a processor and a memory communicatively connected to the at least a processor. The memory instructs the processor to receive, from a first database, a plurality of user data comprising a plurality of metadata. The memory instructs the processor to detach the plurality of metadata for the plurality of user data. The memory instructs the processor to identify a plurality of patient identifiers within the plurality of user data. The memory instructs the processor to generate a plurality of anonymized data and the plurality of metadata as a function of the plurality of patient identifiers and the plurality of metadata. The memory instructs the processor to store the anonymized data and the anonymized metadata separately in a second database. The memory instructs the processor to construct a plurality of anonymized user records as a function of the plurality of anonymized data.

In another aspect, a method for anonymizing user data is disclosed. The method includes receiving, using at least a processor, a plurality of user data comprising a plurality of metadata from a first database. The method includes detaching, using the at least a processor, the plurality of metadata for the plurality of user data. The method includes identifying, using the at least a processor, a plurality of patient identifiers within the plurality of user data and the plurality of metadata. The method includes generating, using the at least a processor, a plurality of anonymized data and anonymized metadata as a function of the plurality of patient identifiers and the plurality of metadata. The method includes storing the anonymized data and the anonymized metadata separately in a second database. The method includes constructing, using the at least a processor, a plurality of anonymized user records as a function of the plurality of anonymized data.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for anonymizing user data;

FIG. 2 is a block diagram of an exemplary machine-learning process;

FIG. 3 is a block diagram of exemplary embodiment of an immutable sequential listing;

FIG. 4 is a diagram of an exemplary embodiment of a neural network;

FIG. 5 is a diagram of an exemplary embodiment of a node of a neural network;

FIG. 6 is an illustration of an exemplary embodiment of fuzzy set comparison;

FIG. 7 is an illustration of an automated one-way tokenization system;

FIG. 8 is an exemplary illustration of a process for generating a medical order using automated one-way tokenization;

FIG. 9 is a flow diagram of an exemplary method for anonymizing user data; and

FIG. 10 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to an apparatus and a method for anonymizing user data is disclosed. The apparatus includes at least a processor and a memory communicatively connected to the at least a processor. The memory instructs the processor to receive, from a first database, a plurality of user data comprising a plurality of metadata. The memory instructs the processor to detach the plurality of metadata for the plurality of user data. The memory instructs the processor to identify a plurality of patient identifiers within the plurality of user data and the plurality of metadata. The memory instructs the processor to generate a plurality of anonymized data and anonymized metadata as a function of the plurality of patient identifiers and the plurality of metadata. The memory instructs the processor to store the anonymized data and the anonymized metadata separately in a second database. The memory instructs the processor to construct a plurality of anonymized user records as a function of the plurality of anonymized data. Exemplary embodiments illustrating aspects of the present disclosure are described below in the context of several specific examples.

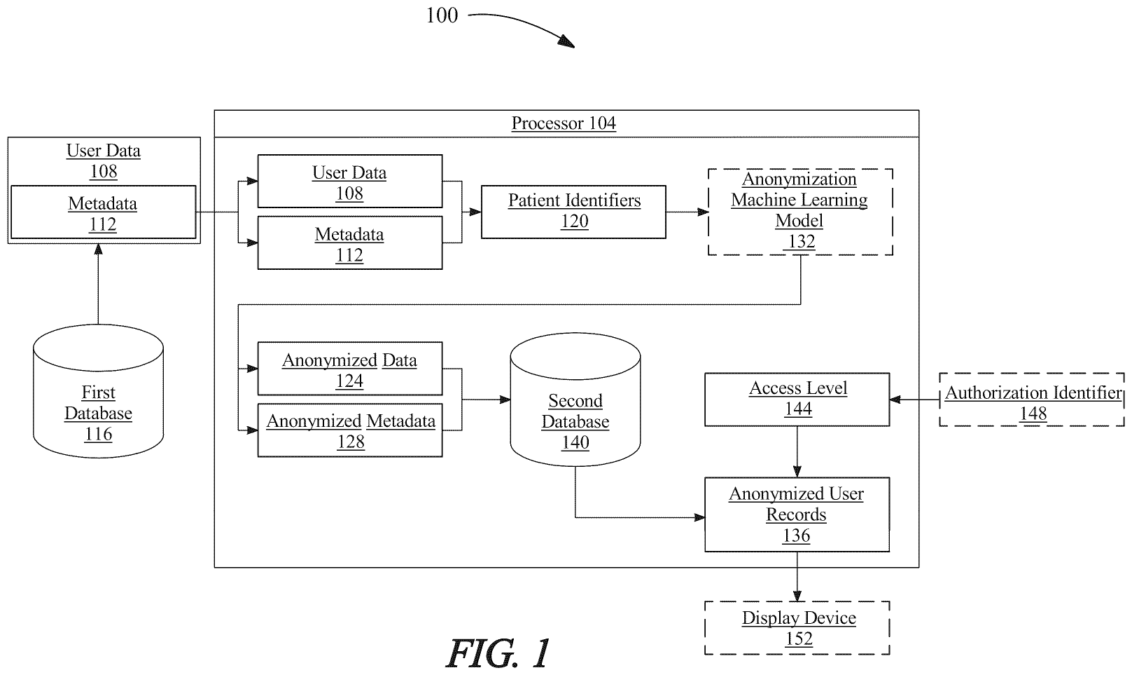

Referring now to FIG. 1, an exemplary embodiment of an apparatus 100 for anonymizing user data is illustrated. Apparatus 100 includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of apparatus 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, apparatus 100 includes a memory. Memory is communicatively connected to processor 104. Memory may contain instructions configuring processor 104 to perform tasks disclosed in this disclosure. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, apparatus, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example, and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, without limitation, via a bus or other facility for intercommunication between elements of a computing device. Communicative connecting may also include indirect connections via, for example, and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, processor 104 is configured to receive a plurality of user data 108. As used in the current disclosure, “user data” is data related to the health and medical history of a user. User data 108 may be used for diagnosing and treating medical conditions, conducting research, managing patient care, and making informed healthcare decisions. User data 108 may include information from a plurality of electronic health records (EHRs). As used in the current disclosure, “electronic health records” are digital records containing a patient's medical history, diagnoses, medications, treatment plans, and other relevant information. EHRs are used by healthcare providers to track and manage patient care. User data 108 may include information such as a plurality of medical imaging data. As used in the current disclosure, “medical imaging data” refers to the visual representations of the internal structures and functions of the human body obtained through various imaging techniques. Medical imaging data may include data associated with X-rays, CT scans, magnetic resonance imaging, ultrasounds, PET scans, nuclear medicine imaging, mammography, fluoroscopy, and the like. User data 108 may include data from blood tests, urine tests, biopsies, and other diagnostic tests are essential for assessing a patient's health and diagnosing diseases. User data 108 may include data associated with the vital signs of the user. This may include data such as blood pressure, heart rate, respiratory rate, and body temperature which are vital for monitoring a patient's condition and overall health. User data 108 may include detailed notes and observations made by healthcare professionals during patient visits, providing additional context to the medical history. User data 108 may be collected in the course of clinical trials, studies, and medical research, which can include genomic data, epidemiological data, and more.

With continued reference to FIG. 1, processor 104 may be configured to receive user data 108 using an application programming interface (API). As used herein, an “application programming interface” is a set of functions that allow applications to access data and interact with external software components, operating systems, or microdevices, such as another web application or computing device. An API may define the methods and data formats that applications can use to request and exchange information. APIs enable seamless integration and functionality between different systems, applications, or platforms. An API may deliver user data 108 to apparatus 100 from a system/application that is associated with a user, medical provider, or other third party custodian of user information. An API may be configured to query for web applications or other websites to retrieve user data 108 or other data associated with the user. An API may be further configured to filter through web applications according to a filter criterion. In this disclosure, “filter criterion” are conditions the web applications must fulfill in order to qualify for API. Web applications may be filtered based off these filter criterion. Filter criterion may include, without limitation, web application dates, web application traffic, web application types, web applications addresses, and the like. Once an API filters through web applications according to a filter criterion, it may select a web application. Processor 104 may transmit, through the API, user data 108 to apparatus 100. API may further automatically fill out user entry fields of the web application with the user credentials in order to gain access to the user data 108. Web applications may include, without limitation, a social media website, an online form, file scanning, email programs, third party websites, governmental websites, or the like.

Continuing to refer to FIG. 1, processor 104 may extract user data 108 from documents or other text received from the user using an optical character recognition system. Optical character recognition or optical character reader (OCR) may be applied upon submission of user data 108 into processor 104 and includes automatic conversion of images of written information (e.g., typed, handwritten or printed text) into machine-encoded text. In some cases, recognition of at least a keyword from an image component may include one or more processes, including without limitation OCR, optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine learning processes. In some cases, intelligent word recognition (IWR) may recognize written text, one word at a time, for instance by employing machine learning processes.

Still referring to FIG. 1, in some cases OCR may be an “offline” process, which analyses a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information can make handwriting recognition more accurate. In some cases, this technology may be referred to as “online” character recognition, dynamic character recognition, real-time character recognition, and intelligent character recognition.

Still referring to FIG. 1, in some cases, OCR processes may employ pre-processing of image component. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to image component to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image component. In some cases, binarization may be required for example if an employed OCR algorithm only works on binary images. In some cases, a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image component.

Still referring to FIG. 1, in some embodiments an OCR process will include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at a same scale as input glyph. Matrix matching may work best with typewritten text.

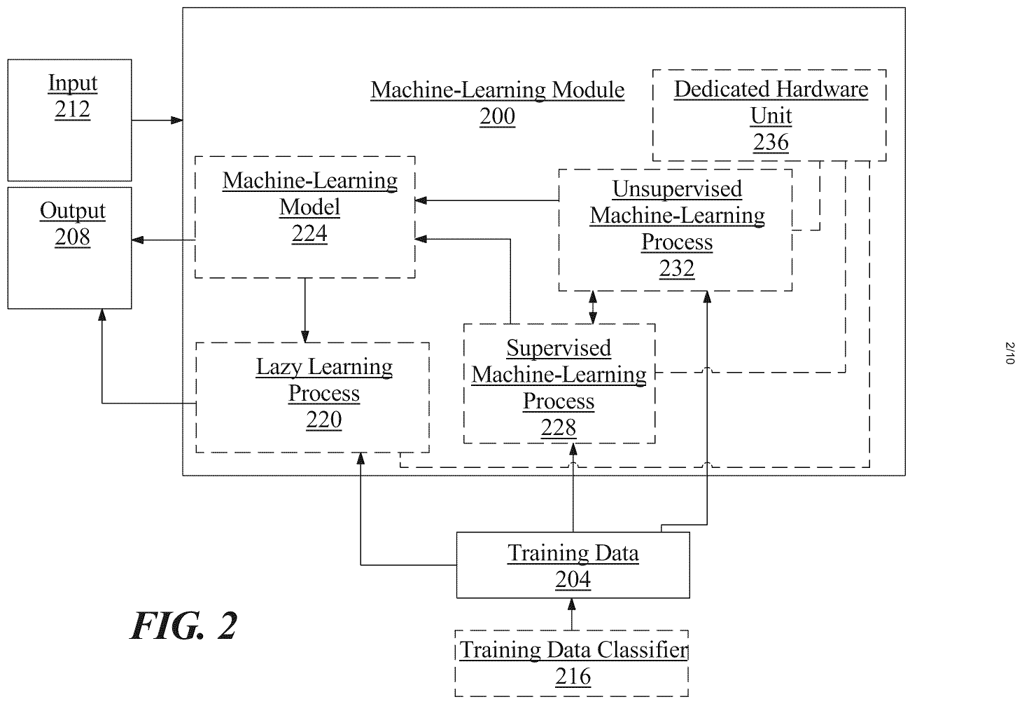

Still referring to FIG. 1, in some embodiments, an OCR process may include a feature extraction process. In some cases, feature extraction may decompose a glyph into features. Exemplary non-limiting features may include corners, edges, lines, closed loops, line direction, line intersections, and the like. In some cases, feature extraction may reduce dimensionality of representation and may make the recognition process computationally more efficient. In some cases, extracted feature can be compared with an abstract vector-like representation of a character, which might reduce to one or more glyph prototypes. General techniques of feature detection in computer vision are applicable to this type of OCR. In some embodiments, machine-learning processes like nearest neighbor classifiers (e.g., k-nearest neighbors algorithm) can be used to compare image features with stored glyph features and choose a nearest match. OCR may employ any machine-learning process described in this disclosure, for example machine-learning processes described with reference to FIG. 2. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

Still referring to FIG. 1, in some cases, OCR may employ a two-pass approach to character recognition. Second pass may include adaptive recognition and use letter shapes recognized with high confidence on a first pass to recognize better remaining letters on the second pass. In some cases, two-pass approach may be advantageous for unusual fonts or low-quality image components where visual verbal content may be distorted. Another exemplary OCR software tool includes OCRopus. OCRopus development is led by German Research Centre for Artificial Intelligence in Kaiserslautern, Germany.

Still referring to FIG. 1, in some cases, OCR may include post-processing. For example, OCR accuracy can be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make us of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

With continued reference to FIG. 1, processor 104 is configured to receive a plurality of user data 108 comprising a plurality of metadata 112. As used in the current disclosure, “metadata” refers to descriptive or informational data that provides details about the user data 108. Metadata 112 may include descriptive metadata, wherein descriptive metadata is configured to describe the content, context, and structure of the data. This may include information such as time, geographic location, medical facility names, medical professional logs, patient names, patient IDs, patient data, X-rays, MRIs, CT Scans, pet scans, ultrasounds, medical images, medical imaging dates, medical imaging technician information, along with any other patient specific data. Metadata 112 may be used to describe records of how the data has been accessed, utilized, or modified over time, aiding in understanding data usage patterns, and optimizing access. In an embodiment, metadata 112 may include Digital Imaging and Communications in Medicine (DICOM) data. Metadata 112 may refers to any form of data that can identify a patient including but not limited to metadata embedded on a medical image, This may include metadata DICOM headers. DICOM Headers may include patient identity revealing information such as contours of head, body/organ profile, etc. In some embodiments, metadata 112 may provide details regarding the management and administration of the data, such as access rights, permissions, versioning, and preservation information. It may include information such as titles, authors, dates, keywords, summaries, and abstracts data or information that is collected, processed, or generated passively in the background without requiring direct input or actions from the user. This data is often gathered by applications, devices, or systems for various purposes, such as improving user experiences, enhancing functionality, or aiding in analytics.

Still referring to FIG. 1, processor 104 may be configured to receive user data 108 from a first database 116. As used in the current disclosure, a “first database” is a database that is configured to store user data 108 and its associated metadata 112. In an embodiment, any past or present versions of any data disclosed herein may be stored within the first database 116 including but not limited to user data 108 and metadata 112. Processor 104 may be communicatively connected with the first database 116. For example, in some cases, the first database 116 may be local to processor 104. Alternatively or additionally, in some cases, the first database 116 may be remote to processor 104 and communicative with processor 104 by way of one or more networks. Network may include, but not limited to, a cloud network, a mesh network, or the like. By way of example, a “cloud-based” system, as that term is used herein, can refer to a system which includes software and/or data which is stored, managed, and/or processed on a network of remote servers hosted in the “cloud,” e.g., via the Internet, rather than on local severs or personal computers. A “mesh network” as used in this disclosure is a local network topology in which the infrastructure processor 104 connects directly, dynamically, and non-hierarchically to as many other computing devices as possible. A “network topology” as used in this disclosure is an arrangement of elements of a communication network The first database 116 may be implemented, without limitation, as a relational database, a key-value retrieval database such as a NOSQL database, or any other format or structure for use as a database that a person skilled in the art would recognize as suitable upon review of the entirety of this disclosure. The first database 116 may alternatively or additionally be implemented using a distributed data storage protocol and/or data structure, such as a distributed hash table or the like. The first database 116 may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. In an embodiment, the disclosure of the first database 116 may be selectively appliable to the second database 140.

With continued reference to FIG. 1, metadata 112 may be detached from the user data 108 using various methods and techniques depending on the type and structure of the data. Detaching metadata 112 from user data 108 is the process of separating identifying or contextual information from the core content or information provided by users. This practice may be useful for safeguarding individual privacy and data anonymity. By removing metadata, such as geolocation, timestamps, or user identifiers, from the associated data, it becomes significantly more challenging to trace the information back to specific individuals, thus reducing the risk of unauthorized surveillance or misuse of personal information. Detaching metadata may protect user privacy and maintaining data integrity in an increasingly interconnected digital. In some cases, data profiling tools automatically analyze the dataset to detach metadata, including statistical summaries (e.g., min, max, mean, standard deviation), data distributions, unique values, and data quality metrics. Profiling tools can help understand the data's characteristics and identify anomalies. Additionally, Software tools may be used to analyze the data to infer its underlying schema or structure. This process involves identifying data types, keys, relationships, and constraints based on patterns and regularities within the dataset. It's especially useful for unstructured or semi-structured data. In an non-limiting embodiment, a natural language processing techniques can be used to extract metadata such as keywords, entities, topics, and sentiment analysis. NLP algorithms can automatically annotate and categorize text, providing valuable metadata about the content. Language processing techniques are discussed in greater detail herein below. In an additional embodiment, machine learning algorithms can be trained to identify and extract specific metadata elements from the dataset. For example, a model could be trained to recognize dates, names, or numerical values within the dataset. For datasets sourced from the web, web scraping techniques, mentioned herein above, can be employed to extract metadata from web pages. This could include extracting information about the source, publication date, author, or any other relevant metadata present on the web. In a third embodiment, metadata 112 may be extracted from the user data 108 utilizing APIs and data catalogs associated with specific datasets or data sources can provide standardized metadata. APIs often offer programmatic access to metadata and dataset information.

With continued reference to FIG. 1, processor 104 identifies a plurality of patient identifiers 120 within the plurality of user data 108 and the metadata 112. As used in the current disclosure, “patient identifiers” refers to information used to identify and distinguish individual patients in healthcare records and systems. Patent identifiers may include any identifiers described in the Health Insurance Portability and Accountability Act (HIPAA). Examples of patient identifiers include the name, address, phone number, email address, phone number, email address, social security number (SSN), national identification number, medical record number, health insurance information, beneficiary information, account numbers, and the like. Patient identifiers may additionally include social media, web addresses, IP addresses, biometric identifiers (i.e. fingerprints, voice prints, and the like.), full face photographic images, any comparable images, and the like. Patient identifiers 120 may include all geographic subdivisions smaller than a state, including street address, city, county, precinct, zip code, and their equivalent geocodes. In an embodiment, patient identifiers 120 may include temporal data. As used in the current disclosure, “temporal data” is data related to the date or time that an event occurred. Temporal data may be any time or date that is a part of the user data 108. Temporal data may include all dates directly related to an individual, including birth date, admission date, discharge date, date of death, and the like. Temporal data may include data associated with the date medical image or a medical test was taken. Temporal data may include dates associated with surgery, procedure, admission, medical professional examinations/appointments, medical tests, symptoms, date of birth, date of death, and the like. Processor 104 may identify a plurality of patient identifiers 120 using a named entity recognitions system, data extraction from an EHR, an identification Machine Learning model, contextual analysis, and the like.

With continued reference to FIG. 1, processor 104 may identify a plurality of patient identifiers 120 using a named entity recognition (NER) system. As used in the current disclosure, a “named entity recognition (NER) system” is software that identifies a plurality of named entities in from text. A NER system may be configured to identify a plurality of named entities from user data 108 its associated metadata 116. Inputs of a NER system may include a user data 108, metadata 112, and the like. The output of a named entity recognition system may include a plurality of named entities associated with known patient identifiers 120. Named entities may include a structured representation of the identified named entities, typically in the form of annotations or tags attached to the original text.

With continued reference to FIG. 1, a NER system may generate a plurality of named entities associated with patient identifiers 120 using a natural language processing model. As used in the current disclosure, a “natural language processing (NLP) model” is a computational model designed to process and understand human language. It leverages techniques from machine learning, linguistics, and computer science to enable computers to comprehend, interpret, and generate natural language text. The NLP model may preprocess the textual data, wherein the input text may include all text contained within the user data 108 its associated metadata 112, or any other data mentioned herein. Preprocessing the input text may involve tasks like tokenization (splitting text into individual words or sub-word units), normalizing the text (lowercasing, removing punctuation, etc.), and encoding the text into a numerical representation suitable for the model. The NLP model may include transformer architecture, wherein the transformers are deep learning models that employ attention mechanisms to capture the relationships between words or sub-word units in a text sequence. They consist of multiple layers of self-attention and feed-forward neural networks. The NLP model may weigh the importance of different words or sub-word units within a text sequence while considering the context. It enables the model to capture dependencies and relationships between words, considering both local and global contexts. This process may be used to identify a plurality of named entities. Language processing model may include a program automatically generated by processor 104 and/or named entity recognition system to produce associations between one or more significant terms extracted from the user data 108 its associated metadata 112 and detect associations, including without limitation mathematical associations, between such significant terms. Associations between language elements, where language elements include for purposes herein extracted significant terms, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted significant term indicates a given category of semantic meaning. As a further example, statistical correlations and/or mathematical associations may include probabilistic formulas or relationships indicating a positive and/or negative association between at least an extracted significant term and/or a given semantic relationship; positive or negative indication may include an indication that a given document is or is not indicating a category semantic relationship. Whether a phrase, sentence, word, or other textual element in the user data 108 its associated metadata 112 constitutes a positive or negative indicator may be determined, in an embodiment, by mathematical associations between detected significant terms, comparisons to phrases and/or words indicating positive and/or negative indicators that are stored in memory at processor 104, or the like.

With continued reference to FIG. 1, processor 104 may identify a plurality of patient identifiers 120 using a pattern recognition process. As used in the current disclosure, a “pattern recognition process” is a process configured to recognize specific patterns or structures commonly associated with patient identifiers. Processor 104 may be configured to define one or more patterns associated with the plurality of patient identifiers 120. These patterns can be based on regular expressions or specific rules that describe the format of each identifier. In an non-limiting example, a pattern for recognizing temporal data might be in the format “MM/DD/YYYY.” In an additional non-limiting example, a pattern for recognizing a SSN might be in the format “XXX-XX-XXXX.” In some cases, the user data 108 may be segmented into discrete sections or fields, such as patient demographics, medical history, and clinical notes. The pattern recognition process may be applied to each section separately to identify patient identifiers 120. The processor 104 may compare the text within each section of the user data 108 to the predefined patterns. It looks for instances where the text matches the expected pattern for a particular identifier. In an non-limiting example, if the pattern recognition algorithm encounters a sequence of numbers in the format “MM/DD/YYYY,” it may identify this as a date of birth.

With continued reference to FIG. 1, processor 104 may identify a plurality of patient identifiers 120 using an identification machine-learning model. As used in the current disclosure, an “identification machine-learning model” is a machine-learning model that is configured to identify a plurality of patient identifiers 120. An identification machine-learning model may be consistent with the machine-learning model described below in FIG. 2. Inputs to the identification machine-learning model may include user data 108, metadata 112, temporal data, medical imaging data, examples of plurality of patient identifiers 120, and the like. Outputs to the identification machine-learning model may include plurality of patient identifiers 120 tailored to the user data 108 its associated metadata 112. identification training data may include a plurality of data entries containing a plurality of inputs that are correlated to a plurality of outputs for training a processor by a machine-learning process. In an embodiment, identification training data may include a plurality of user data 108 its associated metadata 112 correlated to examples of plurality of patient identifiers 120. identification training data may be received from database. identification training data may contain information about user data 108, metadata 112, temporal data, medical imaging data, examples of plurality of patient identifiers 120, and the like. In an embodiment, identification training data may be iteratively updated as a function of the input and output results of past identification machine-learning model or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, processor 104 is configured to generate a plurality of anonymized data 124 and a plurality of anonymized metadata 128 as a function of the plurality of patient identifiers 120. As used in the current disclosure, “anonymized data” is data associated user data, wherein patient identifiers that have been modified or disguised in a way that makes it impossible or impractical to identify the user, while still retaining its usability for specific purposes. As used in the current disclosure, “anonymized metadata” is metadata with each of the patient identifiers obfuscated. Processor 104 is configured to anonymize each patient identifier 120 within the metadata 112 and the user data 108 to generate anonymized data 124 and anonymized metadata 128, respectively. This may include redacting the patient identifiers 120 within the user data 108 and/or metadata 112. Redacting may be done using various methods like blacking out, using placeholders, or applying software tools to mask or replace the sensitive data. In an embodiment, this may involve removing or replacing patient identifiers 120 with pseudonyms and/or generic terms. In an embodiment, any method or procedure for creating anonymized data 124 may be used for creating anonymized metadata 128.

With continued reference to FIG. 1, anonymized data 124 and/or anonymized metadata 128 may include tokenizing the plurality of patient identifiers 120. As used in the current disclosure, “tokenization” is a process where sensitive data elements are replaced with unique tokens or references. Tokenization may rely on a secure mapping or lookup table that links tokens to original data, but this mapping is kept separate and secure. Tokenization of patient identifier 120 may use alphanumeric codes to replace name, geographic locations, temporal data, and the like. In an non-limiting example, a patient identifier 120 associated with a user's name may be replaced by the alphanumeric code of TKN-9876.

With continued reference to FIG. 1, anonymized data 124 and/or anonymized metadata 128 may include aggregating data from a plurality of patients to obfuscate the patient's data. As used in the current disclosure, “aggregating data” is a technique used for anonymizing and summarizing information in a way that conceals individual-level details while still providing valuable insights at a higher level of abstraction. Aggregating data may involve grouping the data into categories or clusters based on common attributes, such as age, location, or other relevant factors. In a non-limiting example, a patient identifier 120 that indicates the user's age of 34 may be aggregated to provide that the user is between the ages of 30-40. In another embodiment, aggregating the patient identifier 120 may include sorting patients into groups of similarly situated patients. They may be similarly situated by demographic factors, symptoms, diagnosis, prognosis, test results, and the like. The patient identifiers 120 of the group may be aggregated for each category, condition, symptom. Common statistical measures may be implemented to provide greater insights into the aggregated data. This may include averages, counts, percentages, mean, median, mode, or other statical measures. In an non limiting example, a medical test result for five patients with who share the same condition may be anonymized by providing the average score among the five patients. The process of combining and summarizing data to create statistical or summary information, thus protecting the privacy of individual data subjects. In another non-limiting example, instead of reporting individual cholesterol levels for patients, an obfuscation process might provide the average cholesterol level for a group of patients within a certain age range. Additionally, generating anonymized data 124 and/or anonymized metadata 128 may involve generalizing the patient identifiers 120. Generalization may involve replacing specific data points with more generalized categories or ranges. In a non-limiting example, instead of listing the exact age of a patient, processor 104 might categorize them as “under 18,” “18-65,” or “65 and older.”

With continued reference to FIG. 1, processor 104 may generate anonymized data 124 and/or anonymized metadata 128 applying a temporal shift to the temporal data. As used in the current disclosure, a “temporal shift” is a process of uniformly altering the temporal dimension of the data, such as timestamps and dates, to make it more challenging to identify individuals and their medical histories. A temporal shift is a technique employed to protect the privacy and confidentiality of patients' health information while still allowing researchers and healthcare professionals to analyze the data for various purposes. While anonymizing data, it's essential to maintain its utility for research or analysis. The temporal shift should not disrupt the chronological order of events in a way that makes the data unusable. A temporal shift may involve shifting the timestamps or dates associated with each data point. The goal is to add a time-based offset to the original timestamps, making it difficult to link the data to specific individuals. For example, you might add or subtract a random number of days, months, or years to each the timestamp. In an embodiment, to implement a temporal shift processor 104 may generate a random number. This number may be between −365 and 365. In some cases, where the latest date is within 1 year from the present date the random number may be limited to negative integers. The random number will be quantified as a number of days. Then the temporal data may be shifted forward or backward a number of days associated with the random number.

With continued reference to FIG. 1, processor 104 may generate anonymized data 124 and/or anonymized metadata 128 using annotation data to sequence a set of temporal data. Temporal data may include gross time identifiers and/or fine grained time identifiers. As used in the current disclosure, a “gross time identifier” is a way to categorize and measure time that uses an increment of 12 hours or more. Examples of gross time identifiers may include days, months, quarters, years, decades, and the like. In an embodiment, processor 104 may employ a gross time identifier when time is based on a date. For instance, the data may be obfuscated by random offsets or a temporal shift. As used in the current disclosure, a “fine grained time identifier” is a way to categorize and measure time that uses an increment of less than 12 hours. Examples of fine time identifiers may include seconds, minutes, hours, and the like. In some cases, a fine grained time identifier may be generated based on clock time. In an embodiment, processor 104 may treat the gross time identifier as anonymized data whereas the fine grained time identifier may be considered non-obfuscated data or personally identifiable data. Alternatively, processor 104 may obfuscate the gross time identifiers by removing the date and replacing it with an arbitrary time identifier (i.e. Day 1, Day 3, Day 7, and the like.) In some embodiments, when generating anonymized data 124, processor 104 may obfuscate the at least one gross time identifier. In some embodiments, obfuscating the at least one gross time identifier may include replacing any gross time identifier with a serialized identifier, removing the gross time identifier, redacting the gross time identifier, blurring the gross time identifier, and the like. In some embodiments, fine grained time identifiers may be retained through the anonymization process.

With continued reference to FIG. 1, processor 104 may obfuscate medical images with serial numbers. This may include sequencing the serial numbers is using hash algorithms. An obfuscated set of serial numbers can be a ordered set of integers. The purpose of this process is to create an ordered sequence of serial numbers from a potentially unorganized set of data. Each item in the dataset may be a assigned a unique serial number. If the items don't have serial numbers already, the processor may assign them or ensure they are readily identifiable in the dataset. The processor selects an appropriate hashing algorithm. Hashing algorithms take an input (in this case, the serial numbers) and produce a fixed-length string of characters, which is typically a hexadecimal or binary representation. Hashing algorithms are described in greater detail herein below. The processor 104 may then apply the chosen hashing algorithm to each serial number. This means that it may process each serial number through the algorithm, and the result is a hash value for each serial number. The hash values may be compared to determine their relative order. The processor may then sort the items based on their hash values. This sorting can be done in ascending or descending order, depending on the desired sequence. The sorted order of items based on their hash values becomes the sequence of serial numbers. This sequence can be stored or used for various purposes, such as data organization, indexing, or identifying items in a specific order.

With continued reference to FIG. 1, processor 104 may generate anonymized data 124 and/or anonymized metadata 128 using a cryptographic system to encrypt the patient identifiers 120. In one embodiment, a cryptographic system is a system that converts data from a first form, known as “plaintext,” which is intelligible when viewed in its intended format, into a second form, known as “ciphertext,” which is not intelligible when viewed in the same way. Ciphertext may be unintelligible in any format unless first converted back to plaintext. In one embodiment, a process of converting plaintext into ciphertext is known as “encryption.” Encryption process may involve the use of a datum, known as an “encryption key,” to alter plaintext. Cryptographic system may also convert ciphertext back into plaintext, which is a process known as “decryption.” Decryption process may involve the use of a datum, known as a “decryption key,” to return the ciphertext to its original plaintext form. In embodiments of cryptographic systems that are “symmetric,” decryption key is essentially the same as encryption key: possession of either key makes it possible to deduce the other key quickly without further secret knowledge. Encryption and decryption keys in symmetric cryptographic systems may be kept secret and shared only with persons or entities that the user of the cryptographic system wishes to be able to decrypt the ciphertext. One example of a symmetric cryptographic system is the Advanced Encryption Standard (“AES”), which arranges plaintext into matrices and then modifies the matrices through repeated permutations and arithmetic operations with an encryption key.

With continued reference to FIG. 1, in embodiments of cryptographic systems that are “asymmetric,” either encryption or decryption key cannot be readily deduced without additional secret knowledge, even given the possession of a corresponding decryption or encryption key, respectively; a common example is a “public key cryptographic system,” in which possession of the encryption key does not make it practically feasible to deduce the decryption key, so that the encryption key may safely be made available to the public. An example of a public key cryptographic system is RSA, in which an encryption key involves the use of numbers that are products of very large prime numbers, but a decryption key involves the use of those very large prime numbers, such that deducing the decryption key from the encryption key requires the practically infeasible task of computing the prime factors of a number which is the product of two very large prime numbers. Another example is elliptic curve cryptography, which relies on the fact that given two points P and Q on an elliptic curve over a finite field, and a definition for addition where A+B=−R, the point where a line connecting point A and point B intersects the elliptic curve, where “0,” the identity, is a point at infinity in a projective plane containing the elliptic curve, finding a number k such that adding P to itself k times results in Q is computationally impractical, given correctly selected elliptic curve, finite field, and P and Q.

With continued reference to FIG. 1, in some embodiments, systems and methods described herein produce cryptographic hashes, also referred to by the equivalent shorthand term “hashes.” A cryptographic hash, as used herein, is a mathematical representation of a lot of data, such as files or blocks in a block chain as described in further detail below; the mathematical representation is produced by a lossy “one-way” algorithm known as a “hashing algorithm.” Hashing algorithm may be a repeatable process; that is, identical lots of data may produce identical hashes each time they are subjected to a particular hashing algorithm. Because hashing algorithm is a one-way function, it may be impossible to reconstruct a lot of data from a hash produced from the lot of data using the hashing algorithm. In the case of some hashing algorithms, reconstructing the full lot of data from the corresponding hash using a partial set of data from the full lot of data may be possible only by repeatedly guessing at the remaining data and repeating the hashing algorithm; it is thus computationally difficult if not infeasible for a single computer to produce the lot of data, as the statistical likelihood of correctly guessing the missing data may be extremely low. However, the statistical likelihood of a computer of a set of computers simultaneously attempting to guess the missing data within a useful timeframe may be higher, permitting mining protocols as described in further detail below.

With continued reference to FIG. 1, in an embodiment, hashing algorithm may demonstrate an “avalanche effect,” whereby even extremely small changes to lot of data produce drastically different hashes. This may thwart attempts to avoid the computational work necessary to recreate a hash by simply inserting a fraudulent datum in data lot, enabling the use of hashing algorithms for “tamper-proofing” data such as data contained in an immutable ledger as described in further detail below. This avalanche or “cascade” effect may be evinced by various hashing processes; persons skilled in the art, upon reading the entirety of this disclosure, will be aware of various suitable hashing algorithms for purposes described herein. Verification of a hash corresponding to a lot of data may be performed by running the lot of data through a hashing algorithm used to produce the hash. Such verification may be computationally expensive, albeit feasible, potentially adding up to significant processing delays where repeated hashing, or hashing of large quantities of data, is required, for instance as described in further detail below. Examples of hashing programs include, without limitation, SHA256, a NIST standard; further current and past hashing algorithms include Winternitz hashing algorithms, various generations of Secure Hash Algorithm (including “SHA-1,” “SHA-2,” and “SHA-3”), “Message Digest” family hashes such as “MD4,” “MD5,” “MD6,” and “RIPEMD,” Keccak, “BLAKE” hashes and progeny (e.g., “BLAKE2,” “BLAKE-256,” “BLAKE-512,” and the like), Message Authentication Code (“MAC”)-family hash functions such as PMAC, OMAC, VMAC, HMAC, and UMAC, Poly1305-AES, Elliptic Curve Only Hash (“ECOH”) and similar hash functions, Fast-Syndrome-based (FSB) hash functions, GOST hash functions, the Grøstl hash function, the HAS-160 hash function, the JH hash function, the RadioGatún hash function, the Skein hash function, the Streebog hash function, the SWIFFT hash function, the Tiger hash function, the Whirlpool hash function, or any hash function that satisfies, at the time of implementation, the requirements that a cryptographic hash be deterministic, infeasible to reverse-hash, infeasible to find collisions, and have the property that small changes to an original message to be hashed will change the resulting hash so extensively that the original hash and the new hash appear uncorrelated to each other. A degree of security of a hash function in practice may depend both on the hash function itself and on characteristics of the message and/or digest used in the hash function. For example, where a message is random, for a hash function that fulfills collision-resistance requirements, a brute-force or “birthday attack” may to detect collision may be on the order of O(2^{n/2}) for n output bits; thus, it may take on the order of 2²⁵⁶ operations to locate a collision in a 512 bit output “Dictionary” attacks on hashes likely to have been generated from a non-random original text can have a lower computational complexity, because the space of entries they are guessing is far smaller than the space containing all random permutations of bits. However, the space of possible messages may be augmented by increasing the length or potential length of a possible message, or by implementing a protocol whereby one or more randomly selected strings or sets of data are added to the message, rendering a dictionary attack significantly less effective.

Continuing to refer to FIG. 1, a “secure proof,” as used in this disclosure, is a protocol whereby an output is generated that demonstrates possession of a secret, such as device-specific secret, without demonstrating the entirety of the device-specific secret; in other words, a secure proof by itself, is insufficient to reconstruct the entire device-specific secret, enabling the production of at least another secure proof using at least a device-specific secret. A secure proof may be referred to as a “proof of possession” or “proof of knowledge” of a secret. Where at least a device-specific secret is a plurality of secrets, such as a plurality of challenge-response pairs, a secure proof may include an output that reveals the entirety of one of the plurality of secrets, but not all of the plurality of secrets; for instance, secure proof may be a response contained in one challenge-response pair. In an embodiment, proof may not be secure; in other words, proof may include a one-time revelation of at least a device-specific secret, for instance as used in a single challenge-response exchange.

With continued reference to FIG. 1, secure proof may include a zero-knowledge proof, which may provide an output demonstrating possession of a secret while revealing none of the secret to a recipient of the output; zero-knowledge proof may be information-theoretically secure, meaning that an entity with infinite computing power would be unable to determine secret from output. Alternatively, zero-knowledge proof may be computationally secure, meaning that determination of secret from output is computationally infeasible, for instance to the same extent that determination of a private key from a public key in a public key cryptographic system is computationally infeasible. Zero-knowledge proof algorithms may generally include a set of two algorithms, a prover algorithm, or “P,” which is used to prove computational integrity and/or possession of a secret, and a verifier algorithm, or “V” whereby a party may check the validity of P. Zero-knowledge proof may include an interactive zero-knowledge proof, wherein a party verifying the proof must directly interact with the proving party; for instance, the verifying and proving parties may be required to be online, or connected to the same network as each other, at the same time. Interactive zero-knowledge proof may include a “proof of knowledge” proof, such as a Schnorr algorithm for proof on knowledge of a discrete logarithm. in a Schnorr algorithm, a prover commits to a randomness r, generates a message based on r, and generates a message adding r to a challenge c multiplied by a discrete logarithm that the prover is able to calculate; verification is performed by the verifier who produced c by exponentiation, thus checking the validity of the discrete logarithm. Interactive zero-knowledge proofs may alternatively or additionally include sigma protocols. Persons skilled in the art, upon reviewing the entirety of this disclosure, will be aware of various alternative interactive zero-knowledge proofs that may be implemented consistently with this disclosure.

With continued reference to FIG. 1, alternatively, zero-knowledge proof may include a non-interactive zero-knowledge, proof, or a proof wherein neither party to the proof interacts with the other party to the proof; for instance, each of a party receiving the proof and a party providing the proof may receive a reference datum which the party providing the proof may modify or otherwise use to perform the proof. As a non-limiting example, zero-knowledge proof may include a succinct non-interactive arguments of knowledge (ZK-SNARKS) proof, wherein a “trusted setup” process creates proof and verification keys using secret (and subsequently discarded) information encoded using a public key cryptographic system, a prover runs a proving algorithm using the proving key and secret information available to the prover, and a verifier checks the proof using the verification key; public key cryptographic system may include RSA, elliptic curve cryptography, ElGamal, or any other suitable public key cryptographic system. Generation of trusted setup may be performed using a secure multiparty computation so that no one party has control of the totality of the secret information used in the trusted setup; as a result, if any one party generating the trusted setup is trustworthy, the secret information may be unrecoverable by malicious parties. As another non-limiting example, non-interactive zero-knowledge proof may include a Succinct Transparent Arguments of Knowledge (ZK-STARKS) zero-knowledge proof. In an embodiment, a ZK-STARKS proof includes a Merkle root of a Merkle tree representing evaluation of a secret computation at some number of points, which may be 1 billion points, plus Merkle branches representing evaluations at a set of randomly selected points of the number of points; verification may include determining that Merkle branches provided match the Merkle root, and that point verifications at those branches represent valid values, where validity is shown by demonstrating that all values belong to the same polynomial created by transforming the secret computation. In an embodiment, ZK-STARKS does not require a trusted setup.

With continued reference to FIG. 1, a zero-knowledge proof may include any other suitable zero-knowledge proof. Zero-knowledge proof may include, without limitation, bulletproofs. Zero-knowledge proof may include a homomorphic public-key cryptography (hPKC)-based proof. Zero-knowledge proof may include a discrete logarithmic problem (DLP) proof. Zero-knowledge proof may include a secure multi-party computation (MPC) proof. Zero-knowledge proof may include, without limitation, an incrementally verifiable computation (IVC). Zero-knowledge proof may include an interactive oracle proof (TOP). Zero-knowledge proof may include a proof based on the probabilistically checkable proof (PCP) theorem, including a linear PCP (LPCP) proof. Persons skilled in the art, upon reviewing the entirety of this disclosure, will be aware of various forms of zero-knowledge proofs that may be used, singly or in combination, consistently with this disclosure.

With continued reference to FIG. 1, in an embodiment, secure proof is implemented using a challenge-response protocol. In an embodiment, this may function as a one-time pad implementation; for instance, a manufacturer or other trusted party may record a series of outputs (“responses”) produced by a device possessing secret information, given a series of corresponding inputs (“challenges”), and store them securely. In an embodiment, a challenge-response protocol may be combined with key generation. A single key may be used in one or more digital signatures as described in further detail below, such as signatures used to receive and/or transfer possession of crypto-currency assets; the key may be discarded for future use after a set period of time. In an embodiment, varied inputs include variations in local physical parameters, such as fluctuations in local electromagnetic fields, radiation, temperature, and the like, such that an almost limitless variety of private keys may be so generated. Secure proof may include encryption of a challenge to produce the response, indicating possession of a secret key. Encryption may be performed using a private key of a public key cryptographic system, or using a private key of a symmetric cryptographic system; for instance, trusted party may verify response by decrypting an encryption of challenge or of another datum using either a symmetric or public-key cryptographic system, verifying that a stored key matches the key used for encryption as a function of at least a device-specific secret. Keys may be generated by random variation in selection of prime numbers, for instance for the purposes of a cryptographic system such as RSA that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem.

With continued reference to FIG. 1, a “digital signature,” as used herein, includes a secure proof of possession of a secret by a signing device, as performed on provided element of data, known as a “message.” A message may include an encrypted mathematical representation of a file or other set of data using the private key of a public key cryptographic system. Secure proof may include any form of secure proof as described above, including without limitation encryption using a private key of a public key cryptographic system as described above. Signature may be verified using a verification datum suitable for verification of a secure proof; for instance, where secure proof is enacted by encrypting message using a private key of a public key cryptographic system, verification may include decrypting the encrypted message using the corresponding public key and comparing the decrypted representation to a purported match that was not encrypted; if the signature protocol is well-designed and implemented correctly, this means the ability to create the digital signature is equivalent to possession of the private decryption key and/or device-specific secret. Likewise, if a message making up a mathematical representation of file is well-designed and implemented correctly, any alteration of the file may result in a mismatch with the digital signature; the mathematical representation may be produced using an alteration-sensitive, reliably reproducible algorithm, such as a hashing algorithm as described above. A mathematical representation to which the signature may be compared may be included with signature, for verification purposes; in other embodiments, the algorithm used to produce the mathematical representation may be publicly available, permitting the easy reproduction of the mathematical representation corresponding to any file.

Still viewing FIG. 1, in some embodiments, digital signatures may be combined with or incorporated in digital certificates. In one embodiment, a digital certificate is a file that conveys information and links the conveyed information to a “certificate authority” that is the issuer of a public key in a public key cryptographic system. Certificate authority in some embodiments contains data conveying the certificate authority's authorization for the recipient to perform a task. The authorization may be the authorization to access a given datum. The authorization may be the authorization to access a given process. In some embodiments, the certificate may identify the certificate authority. The digital certificate may include a digital signature.

With continued reference to FIG. 1, in some embodiments, a third party such as a certificate authority (CA) is available to verify that the possessor of the private key is a particular entity; thus, if the certificate authority may be trusted, and the private key has not been stolen, the ability of an entity to produce a digital signature confirms the identity of the entity and links the file to the entity in a verifiable way. Digital signature may be incorporated in a digital certificate, which is a document authenticating the entity possessing the private key by authority of the issuing certificate authority and signed with a digital signature created with that private key and a mathematical representation of the remainder of the certificate. In other embodiments, digital signature is verified by comparing the digital signature to one known to have been created by the entity that purportedly signed the digital signature; for instance, if the public key that decrypts the known signature also decrypts the digital signature, the digital signature may be considered verified. Digital signature may also be used to verify that the file has not been altered since the formation of the digital signature.

With continued reference to FIG. 1, processor 104 may generate anonymized data 124 and/or anonymized metadata 128 using an anonymization machine-learning model 132. As used in the current disclosure, an “anonymization machine-learning model” is a machine-learning model that is configured to generate anonymized data 124 and/or anonymized metadata 128. An anonymization machine-learning model 132 may be consistent with the machine-learning model described below in FIG. 2. Inputs to the anonymization machine-learning model 132 may include user data 108, metadata 112, temporal data, medical imaging data, plurality of patient identifiers 120, examples of anonymized data 124, examples of anonymized metadata 128, and the like. Outputs to the anonymization machine-learning model 132 may include anonymized data 124 and/or anonymized metadata 128 tailored to the plurality of patient identifiers 120. anonymization training data may include a plurality of data entries containing a plurality of inputs that are correlated to a plurality of outputs for training a processor by a machine-learning process. In an embodiment, anonymization training data may include a plurality of plurality of patient identifiers 120 correlated to examples of anonymized data 124. Alternatively, anonymization training data may include a plurality of plurality of patient identifiers 120 correlated to examples of anonymized metadata 128. Anonymization training data may be received from database. Anonymization training data may contain information about user data 108, metadata 112, temporal data, medical imaging data, plurality of patient identifiers 120, examples of anonymized data 124, examples of anonymized metadata 128, and the like. In an embodiment, anonymization training data may be iteratively updated as a function of the input and output results of past anonymization machine-learning model or any other machine-learning model mentioned throughout this disclosure. The machine-learning model may be performed using, without limitation, linear machine-learning models such as without limitation logistic regression and/or naive Bayes machine-learning models, nearest neighbor machine-learning models such as k-nearest neighbors machine-learning models, support vector machines, least squares support vector machines, fisher's linear discriminant, quadratic machine-learning models, decision trees, boosted trees, random forest machine-learning model, and the like.

With continued reference to FIG. 1, machine learning plays a crucial role in enhancing the function of software for generating a anonymization machine-learning model 132. This may include identifying patterns of within the user data 108 that lead to changes in the capabilities and type of the anonymization machine-learning model 132. By analyzing vast amounts of data related to judicial data, machine learning algorithms can identify patterns, correlations, and dependencies that contribute to a generating the anonymization machine-learning model 132. These algorithms can extract valuable insights from various sources, including text, document, audio, and other multimodal data associated with the user data 108. By applying machine learning techniques, the software can generate the anonymization machine-learning model 132 extremely accurately. Machine learning models may enable the software to learn from past collaborative experiences of the entities and iteratively improve its training data over time.

With continued reference to FIG. 1, processor 104 may be configured to update the training data of the anonymization machine-learning model 132 using user inputs. A anonymization machine-learning model 132 may use user input to update its training data, thereby improving its performance and accuracy. In embodiments, the anonymization machine-learning model 132 may be iteratively updated using input and output results of past iterations of the anonymization machine-learning model 132. The anonymization machine-learning model 132 may then be iteratively retrained using the updated anonymization training data. For instance, and without limitation, anonymization machine-learning model 132 may be trained using first training data from, for example, and without limitation, training data from a user input or database. The anonymization machine-learning model 132 may then be updated by using previous inputs and outputs from the anonymization machine-learning model 132 as second training data to then train a second machine learning model. This process of updating the anonymization machine-learning model 132 and its associated training data may be continuously done to create subsequent anonymization machine-learning models 124 to improve the speed and accuracy of the anonymization machine-learning model 132. When users interact with the software, their actions, preferences, and feedback provide valuable information that can be used to refine and enhance the model. This user input is collected and incorporated into the training data, allowing the machine learning model to learn from real-world interactions and adapt its predictions accordingly. By continually incorporating user input, the model becomes more responsive to user needs and preferences, capturing evolving trends and patterns. This iterative process of updating the training data with user input enables the machine learning model to deliver more personalized and relevant results, ultimately enhancing the overall user experience. The discussion within this paragraph may apply to both the anonymization machine-learning model 132 or any other machine-learning model./classifier discussed herein.

Incorporating the user feedback may include updating the training data by removing or adding correlations of user data to a path or resources as indicated by the feedback. Any machine-learning model as described herein may have the training data updated based on such feedback or data gathered using a web crawler as described above. For example, correlations in training data may be based on outdated information wherein, a web crawler may update such correlations based on more recent resources and information.

With continued reference to FIG. 1, processor 104 may use user feedback to train the machine-learning models and/or classifiers described above. For example, machine-learning models and/or classifiers may be trained using past inputs and outputs of classifier. In some embodiments, if user feedback indicates that an output of machine-learning models and/or classifiers was “bad,” then that output and the corresponding input may be removed from training data used to train machine-learning models and/or classifiers, and/or may be replaced with a value entered by, e.g., another value that represents an ideal output given the input the machine learning model originally received, permitting use in retraining, and adding to training data; in either case, classifier may be retrained with modified training data as described in further detail below. In some embodiments, training data of classifier may include user feedback.

With continued reference to FIG. 1, in some embodiments, an accuracy score may be calculated for the machine-learning model and/or classifier using user feedback. For the purposes of this disclosure, “accuracy score,” is a numerical value concerning the accuracy of a machine-learning model. For example, the accuracy/quality of the outputted anonymization machine-learning model 132 may be averaged to determine an accuracy score. In some embodiments, an accuracy score may be determined for pairing of entities. Accuracy score or another score as described above may indicate a degree of retraining needed for a machine-learning model and/or classifier. Processor 104 may perform a larger number of retraining cycles for a higher number (or lower number, depending on a numerical interpretation used), and/or may collect more training data for such retraining. The discussion within this paragraph and the paragraphs preceding this paragraph may apply to both the anonymization machine-learning model 132 or any other machine-learning model/classifier mentioned herein.

With continued reference to FIG. 1, processor 104 is configured to construct a plurality of anonymized user records 136 as a function of the plurality of anonymized data 124 and/or anonymized metadata 128. As used in the current disclosures, an “anonymized user record” is a data entry that contains information about a user that has been processed to remove or obscure any personally identifiable information. The purpose of anonymizing user records is to protect individuals' privacy while still allowing organizations to analyze and use data for various purposes. In some cases, an anonymized user record 136 may include a plurality. An anonymized user record 136 may contain an unique identifier assigned to each user, typically in the form of an alphanumeric string. An anonymized user record 136 may additionally contain information about the patient's past and current medical conditions, including diagnoses, chronic illnesses, and surgeries, is recorded without revealing the patient's identity. Additionally, details of the medications prescribed to the patient may be included within the anonymized user records 136, including drug names and dosages, are documented without connecting them to the patient's personal information. Anonymized user records 136 might include demographic information such as age, gender, and location. These details may be grouped into broader categories or ranges to prevent identification of specific individuals. In an embodiment, anonymized user records 136 may include one or more medical images that have been anonymized. In certain cases, facial features, tattoos, or other unique identifiers that might appear in the anonymized medical images may be blurred or masked to further ensure patient anonymity. Information about the body part or organ being imaged and the type of examination (e.g., chest X-ray, brain MRI) is retained for clinical context but does not disclose patient details. Additionally, any identifiers of the radiologist or technician involved in the image acquisition are removed or anonymized to prevent the identification of the healthcare provider. In some cases, text annotations or findings written by the radiologist or other healthcare providers may be de-identified or generalized to ensure patient anonymity. In an embodiment, the anonymized user records 136 may be stored on an immutable sequential listing.

With continued reference to FIG. 1, generating anonymized user records 136 may include compressing anonymized user records 136. Data compression for anonymized user records 136 may refer to the process of reducing the size of digital medical data while retaining essential information. This is done to make medical records more manageable, reduce storage requirements, and facilitate faster data transmission and retrieval. Data compression techniques can be applied to various types of medical data, including text-based electronic health records (EHRs), medical images (e.g., X-rays, CT scans), and other healthcare-related data. Compression of the anonymized user records 136 may be done using lossless compression. Lossless compression may reduce file size without any loss of data. It is commonly used for text-based medical records and structured data like EHRs. Lossless compression algorithms, such as ZIP and GZIP, may work by encoding redundant or repetitive data in a more efficient way. When the data is decompressed, it is identical to the original. Compression of the anonymized user records 136 may be done using lossy compression. Lossy compression may reduce file size by discarding some data that is considered less essential. It is often used for medical images and audio files. Lossy compression is suitable when a minor loss of detail is acceptable, as in radiology images. Popular lossy compression methods for medical images include JPEG and JPEG2000. In some cases, medical images within the anonymized user records 136 may be compressed using DICOM (Digital Imaging and Communications in Medicine). DICOM can store medical images in both lossless and lossy formats. In some cases, DICOM-compressed images can reduce file sizes significantly while maintaining diagnostic quality. Alternatively, medical images within the anonymized user records 136 may be compressed using JPEG Compression or JPEG2000. JPEG Compression is commonly used for medical images like X-rays and CT scans. It uses a lossy compression method that can be adjusted to control the degree of compression and image quality. JPEG2000 is another standard used for medical images. It provides both lossless and lossy compression options and is known for its superior image quality compared to traditional JPEG.

With continued reference to FIG. 1, anonymized data 124 and/or anonymized metadata 128 may be stored in a data repositor. In some examples, data stored in a data repository may be compressed. Examples of compression schemes implemented in the data repository may include a standard feature-engineered compression schemes, and a state-of-art compression schemes made possible by current machine learning models that offer better compression than the standard feature-engineered compression scheme. These emerging compression schemes are preferred to their traditional counterparts not for their higher compression without perceptual loss, but also because of the unique properties of some of these compression schemes—they are rich representations that can be used to represent images for models belonging to a specific class of architecture (e.g. transformer). An example of this is the compressed output of a VQGAN or VQ-VAE autoencoder that may compresses a 384×384 image into a 96×96×3 codebook image where the codebook is a set of 8192 vectors. This 4:1 dimensionality reduction coupled with the mapping. VQ-VAE (Vector Quantized Variational Autoencoder) is a specific type of Variational Autoencoder (VAE) that utilizes vector quantization to improve the efficiency and quality of encoding and decoding images or data. It is particularly popular in the field of generative modeling and image synthesis. When compressing images, VQ-VAE can efficiently represent the image content using a much smaller set of discrete codes from the codebook. This results in a highly compressed but visually similar representation of the original image. When decompressing, the decoder uses the codebook to generate a reconstructed images. In an embodiment, data mentioned herein may be compressed using machine learning algorithms; this may include deep image compression, better portable graphic algorithms, WebP, generative adversarial networks, and the like.

Still referring to FIG. 1, processor 104 is be configured to construct anonymized user records 136 from a second database 140. Constructing an anonymized user record 136 from a second database 140 which contains only anonymized data 124 and anonymized metadata 128 involves aggregating and organizing information in such a way that the individual's identity is protected. This process is essential for data privacy and security, particularly in situations where personal information needs to be shared or analyzed without revealing the identities of the individuals involved. Anonymized data 126 and anonymized metadata 128 may be stored separately within second database 140. The plurality of anonymized data 124 and anonymized metadata 128 may be stored in a database separate from the non-obfuscated user data 108 and metadata 112. In some cases, processor 104 may be communicatively connected to a sandbox database. As used in the current disclosure, a “sandbox database” is a dedicated and isolated database that may access allows for monitoring and logging the use of both anonymized and non-anonymized data. Sandbox database may include any anonymized or non-anonymized information disclosed in this disclosure. In an embodiment, the disclosure of the second database 140 may be selectively include terms and embodiments discussed in relation to the first database 116. A sandbox database may store user data 108, metadata 112, anonymized data 120, anonymized metadata 124, and the like.

With continued reference to FIG. 1, processor 104 may determine an access level 144 of a user as using an authorization identifier. As used in the current disclosure, an “access level” is a level of security. Access level 144 may ensure that sensitive patient information is handled securely and in compliance with legal and ethical standards. These access levels may be designed to control who can view, edit, or transmit user data 108 or anonymized user records 136. Access levels 144 may vary slightly depending on the specific healthcare organization. Examples of access levels 144 may include but are not limited to “No Access,” “Full Anonymized Read-only Access,” “Partially Anonymized Read-only Access,” “Partially Anonymized Read-Write Access,” “Non-anonymized Read-only Access,” “Non-anonymized Read-write Access,” and the like. Processor 104 may construct a plurality of anonymized user records 136 as a function of the access level 144 of the user. Processor 104 may construct an anonymized user record 136 as a function of the user's request. In an non-limiting example, a user may place a request for anonymized user records 136 they may then provide apparatus 100 the authorization identifier 148. Processor 104 may then generate an anonymized user record 136 automatically as a function of the user's request. The anonymized user record 136 that is generated may be tailored to the users access level 144. For example, processor 104 may generate a fully anonymized version of the user records if the users has a first access level. Alternatively, processor 104 may generate a non-anonymized version of the user records if the user has a second access level. In some cases, the level of anonymization of the anonymized user records 136 may be changed as the user's access level changes. For example, if a user has a third access level a patients names may be anonymized while the dates and locations remain non-obfuscated. Processor 104 may construct the anonymized user record 136 from a first database 116 and/or a second database 140. In some cases, the constructed anonymized user record 136 may be constructed from only anonymized data 124 and anonymized metadata 128. In other cases, the constructed anonymized user records 136 may be constructed from a combination of anonymized data 124, anonymized metadata 128, user data 108, and metadata 112. This may include information such as revealing the patients geographic location while maintaining their anonymity.

With continued reference to FIG. 1, processor 104 may generate different instantiations of data depending on the access level of the user. As a non-limiting example, processor may generate a first instantiation if the user has a first access level. First instantiation may include all of anonymized data 124 and anonymized metadata 128, where the anonymized data 124 and anonymized metadata 128 has been recombined. As a non-limiting example, processor may generate a second instantiation if the user has a second access level. Second instantiation may include separated anonymized data 124 and anonymized metadata 128. As a non-limiting example, processor may generate a third instantiation if the user has a third access level. Third instantiation may include only a subset of anonymized text data without any image data. As a non-limiting example, processor may generate a fourth instantiation if the user has a fourth access level. Fourth instantiation may include non-anonymized data.

With continued reference to FIG. 1, the access level 144 of a user may be determined as a function of an authorization identifier 148. As used in the current disclosure, an “authorization identifier” is a unique code or token used to verify and grant access or permission to a specific resource, system, or service. It may serve as a means to authenticate and authorize individuals, applications, or entities to perform certain actions or access particular information. When a user interacts with the anonymized user records 136, they may be required to provide this identifier to verify their identity and access level. An authorization identifier 148 may play a crucial role in ensuring that only authorized individuals or systems can access and use the user data 108 or anonymized user records 136, thus enhancing the security and privacy of the interactions. An authorization identifier 148 may be used to regulate access to user data 108 or anonymized user records 136. In some cases, a user may be required to possess a knowledge factor in order to access the user data 108 or anonymized user records 136, wherein a knowledge factor may include knowledge of a code, such as an alphanumeric code. The authorization identifier 148 can be in the form of a token, such as an API key, authentication token, or a unique session identifier. These tokens are issued during the authentication process and are used to access the user data 108 or anonymized user records 136 securely. In some cases the authorization identifier 148 may be a part of a multi-factor authentication system, where users need to provide additional verification methods, such as a password or biometrics, along with the authorization identifier 148, to access the user records.

Still referring to FIG. 1, an authorization identifier 148 may include a decentralized token. As used in the current disclosure, a “decentralized token” is a digital asset that operates on a decentralized network, typically utilizing an immutable sequential listing. A decentralized token may utilizes a decentralized network of nodes for validation and consensus, eliminating the need for a central authority and enhancing security through the redundancy and distributed nature of the network. The token's security is maintained through cryptographic algorithms, ensuring authentication, confidentiality, and integrity of transactions and data, bolstering trust and transparency within the decentralized ecosystem. A decentralized token may be governed by a distributed and often immutable sequential listing, where transactions and ownership are recorded and verified through a consensus mechanism involving a network of participants. A decentralized token may be distributed to users according to their access level. Each decentralized token may be unique to the user it was assigned to.

Still referring to FIG. 1, processor 104 may be configured to display a anonymized user records 136 using a display device 152. As used in the current disclosure, a “display device” is a device that is used to display a plurality of data and other digital content. A display device 152 may include a user interface. A “user interface,” as used herein, is a means by which a user and a computer system interact; for example through the use of input devices and software. A user interface may include a graphical user interface (GUI), command line interface (CLI), menu-driven user interface, touch user interface, voice user interface (VUI), form-based user interface, any combination thereof, and the like. A user interface may include a smartphone, smart tablet, desktop, or laptop operated by the user. In an embodiment, the user interface may include a graphical user interface. A “graphical user interface (GUI),” as used herein, is a graphical form of user interface that allows users to interact with electronic devices. In some embodiments, GUI may include icons, menus, other visual indicators, or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pulldown menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access. Information contained in user interface may be directly influenced using graphical control elements such as widgets. A “widget,” as used herein, is a user control element that allows a user to control and change the appearance of elements in the user interface. In this context a widget may refer to a generic GUI element such as a check box, button, or scroll bar to an instance of that element, or to a customized collection of such elements used for a specific function or application (such as a dialog box for users to customize their computer screen appearances). User interface controls may include software components that a user interacts with through direct manipulation to read or edit information displayed through user interface. Widgets may be used to display lists of related items, navigate the system using links, tabs, and manipulate data using check boxes, radio boxes, and the like.

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm instantiated in hardware or software logic, data structures, and/or functions that will be performed by a computing device/module to produce outputs 208 given data provided as inputs 212; this is in contrast to a non-machine learning software program where the commands to be executed are determined in advance by a user and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure. As a non-limiting illustrative example a plurality of patient identifiers as inputs correlated to examples of anonymized data as outputs.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to classify each patient identifier in to an access level.

With further reference to FIG. 2, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Still referring to FIG. 2, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may identified as having poor quality data, where “poor quality” is defined as having a signal to noise ratio below a threshold value.

As a non-limiting example, and with further reference to FIG. 2, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 2, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units

In some embodiments, and with continued reference to FIG. 2, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include a plurality of patient identifiers as described above as inputs, examples of anonymized data as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

With further reference to FIG. 2, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 2, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 232 may not require a response variable; unsupervised processes 232 may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 2, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 2, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 2, retraining and/or additional training may be performed using any process for training described above, using any currently or previously deployed version of a machine-learning model and/or algorithm as a starting point. Training data for retraining may be collected, preconditioned, sorted, classified, sanitized, or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 2, one or more processes or algorithms described above may be performed by at least a dedicated hardware unit 236. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 236 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 236 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 236 to perform one or more operations described herein, such as evaluation of model and/or algorithm outputs, one-time or iterative updates to parameters, coefficients, weights, and/or biases, and/or any other operations such as vector and/or matrix operations as described in this disclosure.

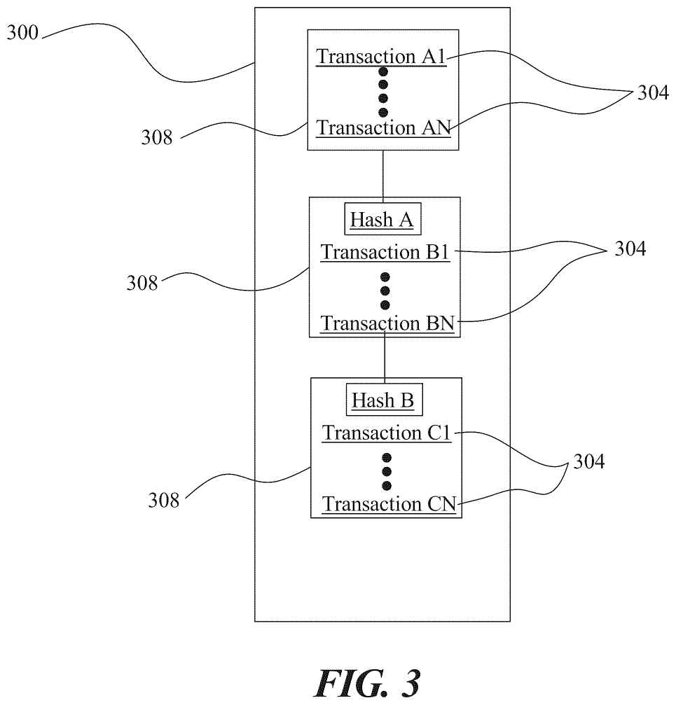

Referring now to FIG. 3, an exemplary embodiment of an immutable sequential listing is illustrated. An “immutable sequential listing,” as used in this disclosure, is a data structure that places data entries in a fixed sequential arrangement, such as a temporal sequence of entries and/or blocks thereof, where the sequential arrangement, once established, cannot be altered, or reordered. An immutable sequential listing may be, include and/or implement an immutable ledger, where data entries that have been posted to the immutable sequential listing cannot be altered. Data elements are listing in immutable sequential listing; data elements may include any form of data, including textual data, image data, encrypted data, cryptographically hashed data, and the like. Data elements may include, without limitation, one or more at least a digitally signed assertions. In one embodiment, a digitally signed assertion 304 is a collection of textual data signed using a secure proof as described in further detail below; secure proof may include, without limitation, a digital signature as described above. Collection of textual data may contain any textual data, including without limitation American Standard Code for Information Interchange (ASCII), Unicode, or similar computer-encoded textual data, any alphanumeric data, punctuation, diacritical mark, or any character or other marking used in any writing system to convey information, in any form, including any plaintext or cyphertext data; in an embodiment, collection of textual data may be encrypted, or may be a hash of other data, such as a root or node of a Merkle tree or hash tree, or a hash of any other information desired to be recorded in some fashion using a digitally signed assertion 304. In an embodiment, collection of textual data states that the owner of a certain transferable item represented in a digitally signed assertion 304 register is transferring that item to the owner of an address. A digitally signed assertion 304 may be signed by a digital signature created using the private key associated with the owner's public key, as described above.

Still referring to FIG. 3, a digitally signed assertion 304 may describe a transfer of virtual currency, such as crypto-currency as described below. The virtual currency may be a digital currency. Item of value may be a transfer of trust, for instance represented by a statement vouching for the identity or trustworthiness of the first entity. Item of value may be an interest in a fungible negotiable financial instrument representing ownership in a public or private corporation, a creditor relationship with a governmental body or a corporation, rights to ownership represented by an option, derivative financial instrument, commodity, debt-backed security such as a bond or debenture or other security as described in further detail below. A resource may be a physical machine e.g. a ride share vehicle or any other asset. A digitally signed assertion 304 may describe the transfer of a physical good; for instance, a digitally signed assertion 304 may describe the sale of a product. In some embodiments, a transfer nominally of one item may be used to represent a transfer of another item; for instance, a transfer of virtual currency may be interpreted as representing a transfer of an access right; conversely, where the item nominally transferred is something other than virtual currency, the transfer itself may still be treated as a transfer of virtual currency, having value that depends on many potential factors including the value of the item nominally transferred and the monetary value attendant to having the output of the transfer moved into a particular user's control. The item of value may be associated with a digitally signed assertion 304 by means of an exterior protocol, such as the COLORED COINS created according to protocols developed by The Colored Coins Foundation, the MASTERCOIN protocol developed by the Mastercoin Foundation, or the ETHEREUM platform offered by the Stiftung Ethereum Foundation of Baar, Switzerland, the Thunder protocol developed by Thunder Consensus, or any other protocol.

Still referring to FIG. 3, in one embodiment, an address is a textual datum identifying the recipient of virtual currency or another item of value in a digitally signed assertion 304. In some embodiments, address is linked to a public key, the corresponding private key of which is owned by the recipient of a digitally signed assertion 304. For instance, address may be the public key. Address may be a representation, such as a hash, of the public key. Address may be linked to the public key in memory of a computing device, for instance via a “wallet shortener” protocol. Where address is linked to a public key, a transferee in a digitally signed assertion 304 may record a subsequent a digitally signed assertion 304 transferring some or all of the value transferred in the first a digitally signed assertion 304 to a new address in the same manner. A digitally signed assertion 304 may contain textual information that is not a transfer of some item of value in addition to, or as an alternative to, such a transfer. For instance, as described in further detail below, a digitally signed assertion 304 may indicate a confidence level associated with a distributed storage node as described in further detail below.

In an embodiment, and still referring to FIG. 3 immutable sequential listing records a series of at least a posted content in a way that preserves the order in which the at least a posted content took place. Temporally sequential listing may be accessible at any of various security settings; for instance, and without limitation, temporally sequential listing may be readable and modifiable publicly, may be publicly readable but writable only by entities and/or devices having access privileges established by password protection, confidence level, or any device authentication procedure or facilities described herein, or may be readable and/or writable only by entities and/or devices having such access privileges. Access privileges may exist in more than one level, including, without limitation, a first access level or community of permitted entities and/or devices having ability to read, and a second access level or community of permitted entities and/or devices having ability to write; first and second community may be overlapping or non-overlapping. In an embodiment, posted content and/or immutable sequential listing may be stored as one or more zero knowledge sets (ZKS), Private Information Retrieval (PIR) structure, or any other structure that allows checking of membership in a set by querying with specific properties. Such database may incorporate protective measures to ensure that malicious actors may not query the database repeatedly in an effort to narrow the members of a set to reveal uniquely identifying information of a given posted content.

Still referring to FIG. 3, immutable sequential listing may preserve the order in which the at least a posted content took place by listing them in chronological order; alternatively or additionally, immutable sequential listing may organize digitally signed assertions 304 into sub-listings 308 such as “blocks” in a blockchain, which may be themselves collected in a temporally sequential order; digitally signed assertions 304 within a sub-listing 308 may or may not be temporally sequential. The ledger may preserve the order in which at least a posted content took place by listing them in sub-listings 308 and placing the sub-listings 308 in chronological order. The immutable sequential listing may be a distributed, consensus-based ledger, such as those operated according to the protocols promulgated by Ripple Labs, Inc., of San Francisco, Calif., or the Stellar Development Foundation, of San Francisco, Calif, or of Thunder Consensus. In some embodiments, the ledger is a secured ledger; in one embodiment, a secured ledger is a ledger having safeguards against alteration by unauthorized parties. The ledger may be maintained by a proprietor, such as a system administrator on a server, that controls access to the ledger; for instance, the user account controls may allow contributors to the ledger to add at least a posted content to the ledger but may not allow any users to alter at least a posted content that have been added to the ledger. In some embodiments, ledger is cryptographically secured; in one embodiment, a ledger is cryptographically secured where each link in the chain contains encrypted or hashed information that makes it practically infeasible to alter the ledger without betraying that alteration has taken place, for instance by requiring that an administrator or other party sign new additions to the chain with a digital signature. Immutable sequential listing may be incorporated in, stored in, or incorporate, any suitable data structure, including without limitation any database, datastore, file structure, distributed hash table, directed acyclic graph or the like. In some embodiments, the timestamp of an entry is cryptographically secured and validated via trusted time, either directly on the chain or indirectly by utilizing a separate chain. In one embodiment the validity of timestamp is provided using a time stamping authority as described in the RFC 3161 standard for trusted timestamps, or in the ANSI ASC x9.95 standard. In another embodiment, the trusted time ordering is provided by a group of entities collectively acting as the time stamping authority with a requirement that a threshold number of the group of authorities sign the timestamp.

In some embodiments, and with continued reference to FIG. 3, immutable sequential listing, once formed, may be inalterable by any party, no matter what access rights that party possesses. For instance, immutable sequential listing may include a hash chain, in which data is added during a successive hashing process to ensure non-repudiation. Immutable sequential listing may include a block chain. In one embodiment, a block chain is immutable sequential listing that records one or more new at least a posted content in a data item known as a sub-listing 308 or “block.” An example of a block chain is the BITCOIN block chain used to record BITCOIN transactions and values. Sub-listings 308 may be created in a way that places the sub-listings 308 in chronological order and link each sub-listing 308 to a previous sub-listing 308 in the chronological order so that any computing device may traverse the sub-listings 308 in reverse chronological order to verify any at least a posted content listed in the block chain. Each new sub-listing 308 may be required to contain a cryptographic hash describing the previous sub-listing 308. In some embodiments, the block chain contains a single first sub-listing 308 sometimes known as a “genesis block.”

Still referring to FIG. 3, the creation of a new sub-listing 308 may be computationally expensive; for instance, the creation of a new sub-listing 308 may be designed by a “proof of work” protocol accepted by all participants in forming the immutable sequential listing to take a powerful set of computing devices a certain period of time to produce. Where one sub-listing 308 takes less time for a given set of computing devices to produce the sub-listing 308 protocol may adjust the algorithm to produce the next sub-listing 308 so that it will require more steps; where one sub-listing 308 takes more time for a given set of computing devices to produce the sub-listing 308 protocol may adjust the algorithm to produce the next sub-listing 308 so that it will require fewer steps. As an example, protocol may require a new sub-listing 308 to contain a cryptographic hash describing its contents; the cryptographic hash may be required to satisfy a mathematical condition, achieved by having the sub-listing 308 contain a number, called a nonce, whose value is determined after the fact by the discovery of the hash that satisfies the mathematical condition. Continuing the example, the protocol may be able to adjust the mathematical condition so that the discovery of the hash describing a sub-listing 308 and satisfying the mathematical condition requires more or less steps, depending on the outcome of the previous hashing attempt. Mathematical condition, as an example, might be that the hash contains a certain number of leading zeros and a hashing algorithm that requires more steps to find a hash containing a greater number of leading zeros, and fewer steps to find a hash containing a lesser number of leading zeros. In some embodiments, production of a new sub-listing 308 according to the protocol is known as “mining.” The creation of a new sub-listing 308 may be designed by a “proof of stake” protocol as will be apparent to those skilled in the art upon reviewing the entirety of this disclosure.

Continuing to refer to FIG. 3, in some embodiments, protocol also creates an incentive to mine new sub-listings 308. The incentive may be financial; for instance, successfully mining a new sub-listing 308 may result in the person or entity that mines the sub-listing 308 receiving a predetermined amount of currency. The currency may be fiat currency. Currency may be cryptocurrency as defined below. In other embodiments, incentive may be redeemed for particular products or services; the incentive may be a gift certificate with a particular business, for instance. In some embodiments, incentive is sufficiently attractive to cause participants to compete for the incentive by trying to race each other to the creation of sub-listings 308 Each sub-listing 308 created in immutable sequential listing may contain a record or at least a posted content describing one or more addresses that receive an incentive, such as virtual currency, as the result of successfully mining the sub-listing 308.

With continued reference to FIG. 3, where two entities simultaneously create new sub-listings 308, immutable sequential listing may develop a fork; protocol may determine which of the two alternate branches in the fork is the valid new portion of the immutable sequential listing by evaluating, after a certain amount of time has passed, which branch is longer. “Length” may be measured according to the number of sub-listings 308 in the branch. Length may be measured according to the total computational cost of producing the branch. Protocol may treat only at least a posted content contained the valid branch as valid at least a posted content. When a branch is found invalid according to this protocol, at least a posted content registered in that branch may be recreated in a new sub-listing 308 in the valid branch; the protocol may reject “double spending” at least a posted content that transfer the same virtual currency that another at least a posted content in the valid branch has already transferred. As a result, in some embodiments the creation of fraudulent at least a posted content requires the creation of a longer immutable sequential listing branch by the entity attempting the fraudulent at least a posted content than the branch being produced by the rest of the participants; as long as the entity creating the fraudulent at least a posted content is likely the only one with the incentive to create the branch containing the fraudulent at least a posted content, the computational cost of the creation of that branch may be practically infeasible, guaranteeing the validity of all at least a posted content in the immutable sequential listing.

Still referring to FIG. 3, additional data linked to at least a posted content may be incorporated in sub-listings 308 in the immutable sequential listing; for instance, data may be incorporated in one or more fields recognized by block chain protocols that permit a person or computer forming a at least a posted content to insert additional data in the immutable sequential listing. In some embodiments, additional data is incorporated in an unspendable at least a posted content field. For instance, the data may be incorporated in an OP RETURN within the BITCOIN block chain. In other embodiments, additional data is incorporated in one signature of a multi-signature at least a posted content. In an embodiment, a multi-signature at least a posted content is at least a posted content to two or more addresses. In some embodiments, the two or more addresses are hashed together to form a single address, which is signed in the digital signature of the at least a posted content. In other embodiments, the two or more addresses are concatenated. In some embodiments, two or more addresses may be combined by a more complicated process, such as the creation of a Merkle tree or the like. In some embodiments, one or more addresses incorporated in the multi-signature at least a posted content are typical crypto-currency addresses, such as addresses linked to public keys as described above, while one or more additional addresses in the multi-signature at least a posted content contain additional data related to the at least a posted content; for instance, the additional data may indicate the purpose of the at least a posted content, aside from an exchange of virtual currency, such as the item for which the virtual currency was exchanged. In some embodiments, additional information may include network statistics for a given node of network, such as a distributed storage node, e.g. the latencies to nearest neighbors in a network graph, the identities or identifying information of neighboring nodes in the network graph, the trust level and/or mechanisms of trust (e.g. certificates of physical encryption keys, certificates of software encryption keys, (in non-limiting example certificates of software encryption may indicate the firmware version, manufacturer, hardware version and the like), certificates from a trusted third party, certificates from a decentralized anonymous authentication procedure, and other information quantifying the trusted status of the distributed storage node) of neighboring nodes in the network graph, IP addresses, GPS coordinates, and other information informing location of the node and/or neighboring nodes, geographically and/or within the network graph. In some embodiments, additional information may include history and/or statistics of neighboring nodes with which the node has interacted. In some embodiments, this additional information may be encoded directly, via a hash, hash tree or other encoding.

With continued reference to FIG. 3, in some embodiments, virtual currency is traded as a crypto-currency. In one embodiment, a crypto-currency is a digital, currency such as Bitcoins, Peercoins, Namecoins, and Litecoins. Crypto-currency may be a clone of another crypto-currency. The crypto-currency may be an “alt-coin.” Crypto-currency may be decentralized, with no particular entity controlling it; the integrity of the crypto-currency may be maintained by adherence by its participants to established protocols for exchange and for production of new currency, which may be enforced by software implementing the crypto-currency. Crypto-currency may be centralized, with its protocols enforced or hosted by a particular entity. For instance, crypto-currency may be maintained in a centralized ledger, as in the case of the XRP currency of Ripple Labs, Inc., of San Francisco, Calif. In lieu of a centrally controlling authority, such as a national bank, to manage currency values, the number of units of a particular crypto-currency may be limited; the rate at which units of crypto-currency enter the market may be managed by a mutually agreed-upon process, such as creating new units of currency when mathematical puzzles are solved, the degree of difficulty of the puzzles being adjustable to control the rate at which new units enter the market. Mathematical puzzles may be the same as the algorithms used to make productions of sub-listings 308 in a block chain computationally challenging; the incentive for producing sub-listings 308 may include the grant of new crypto-currency to the miners. Quantities of crypto-currency may be exchanged using at least a posted content as described above.

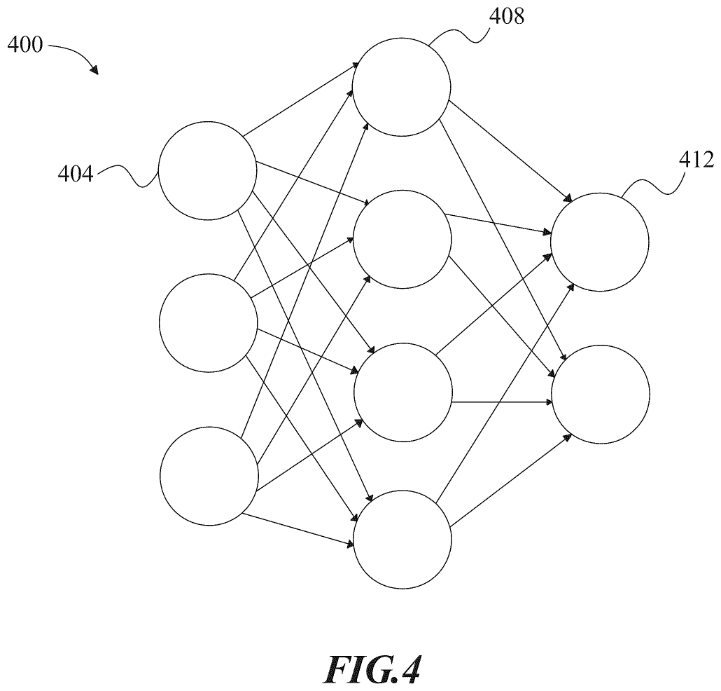

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

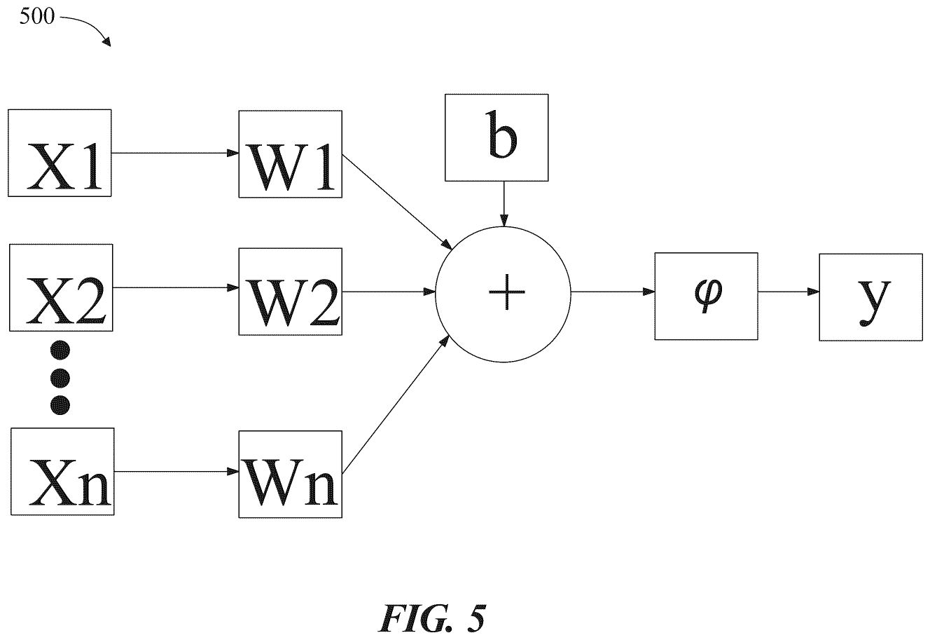

Referring now to FIG. 5, an exemplary embodiment of a node of a neural network is illustrated. A node may include, without limitation, a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function co, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

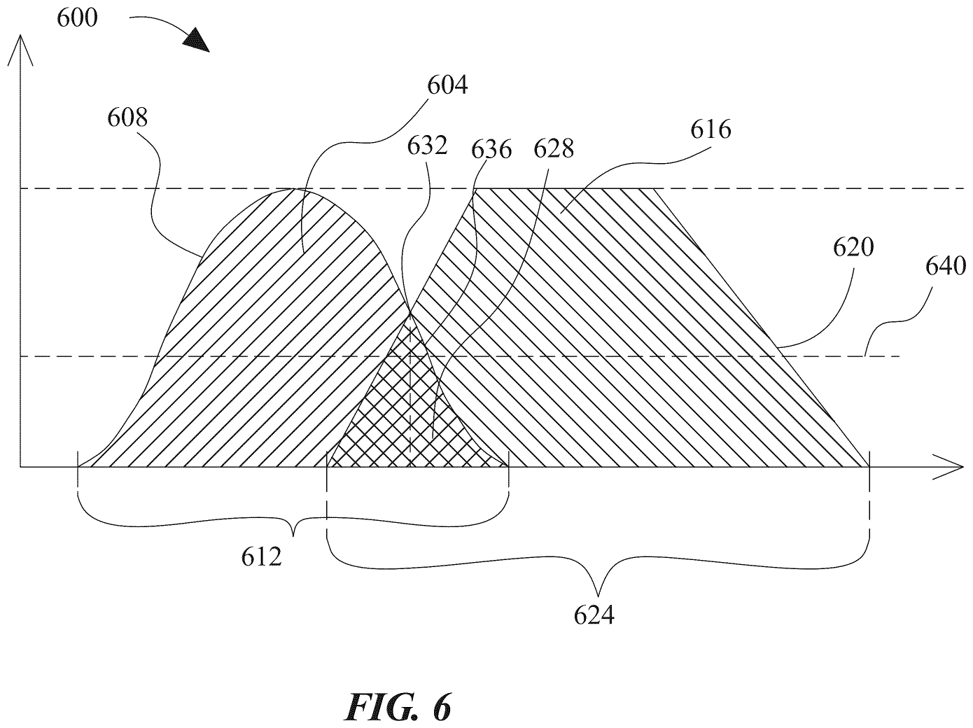

Now referring to FIG. 6, an exemplary embodiment of fuzzy set comparison 600 is illustrated. In a non-limiting embodiment, the fuzzy set comparison. In a non-limiting embodiment, fuzzy set comparison 600 may be consistent with fuzzy set comparison in FIG. 1. In another non-limiting the fuzzy set comparison 600 may be consistent with the name/version matching as described herein. For example and without limitation, the parameters, weights, and/or coefficients of the membership functions may be tuned using any machine-learning methods for the name/version matching as described herein. In another non-limiting embodiment, the fuzzy set may represent user data 108 and patient identifiers 120 from FIG. 1.

Alternatively or additionally, and still referring to FIG. 6, fuzzy set comparison 600 may be generated as a function of determining the data compatibility threshold. The compatibility threshold may be determined by a computing device. In some embodiments, a computing device may use a logic comparison program, such as, but not limited to, a fuzzy logic model to determine the compatibility threshold and/or version authenticator. Each such compatibility threshold may be represented as a value for a posting variable representing the compatibility threshold, or in other words a fuzzy set as described above that corresponds to a degree of compatibility and/or allowability as calculated using any statistical, machine-learning, or other method that may occur to a person skilled in the art upon reviewing the entirety of this disclosure. In some embodiments, determining the compatibility threshold and/or version authenticator may include using a linear regression model. A linear regression model may include a machine learning model. A linear regression model may map statistics such as, but not limited to, frequency of the same range of version numbers, and the like, to the compatibility threshold and/or version authenticator. In some embodiments, determining the compatibility threshold of any posting may include using a classification model. A classification model may be configured to input collected data and cluster data to a centroid based on, but not limited to, frequency of appearance of the range of versioning numbers, linguistic indicators of compatibility and/or allowability, and the like. Centroids may include scores assigned to them such that the compatibility threshold may each be assigned a score. In some embodiments, a classification model may include a K-means clustering model. In some embodiments, a classification model may include a particle swarm optimization model. In some embodiments, determining a compatibility threshold may include using a fuzzy inference engine. A fuzzy inference engine may be configured to map one or more compatibility threshold using fuzzy logic. In some embodiments, a plurality of computing devices may be arranged by a logic comparison program into compatibility arrangements. A “compatibility arrangement” as used in this disclosure is any grouping of objects and/or data based on skill level and/or output score. Membership function coefficients and/or constants as described above may be tuned according to classification and/or clustering algorithms. For instance, and without limitation, a clustering algorithm may determine a Gaussian or other distribution of questions about a centroid corresponding to a given compatibility threshold and/or version authenticator, and an iterative or other method may be used to find a membership function, for any membership function type as described above, that minimizes an average error from the statistically determined distribution, such that, for instance, a triangular or Gaussian membership function about a centroid representing a center of the distribution that most closely matches the distribution. Error functions to be minimized, and/or methods of minimization, may be performed without limitation according to any error function and/or error function minimization process and/or method as described in this disclosure.

Still referring to FIG. 6, inference engine may be implemented according to input of user data 108 and patient identifiers 120. For instance, an acceptance variable may represent a first measurable value pertaining to the classification of user data 108 to patient identifiers 120. Continuing the example, an output variable may represent patient identifiers 120 associated with the user. In an embodiment, user data 108 and/or patient identifiers 120 may be represented by their own fuzzy set. In other embodiments, the classification of the data into patient identifiers 120 may be represented as a function of the intersection two fuzzy sets as shown in FIG. 6, An inference engine may combine rules, such as any semantic versioning, semantic language, version ranges, and the like thereof. The degree to which a given input function membership matches a given rule may be determined by a triangular norm or “T-norm” of the rule or output function with the input function, such as min (a, b), product of a and b, drastic product of a and b, Hamacher product of a and b, or the like, satisfying the rules of commutativity (T(a, b)=T(b, a)), monotonicity: (T(a, b)≤T(c, d) if a≤c and b≤d), (associativity: T(a, T(b, c))=T(T(a, b), c)), and the requirement that the number 1 acts as an identity element. Combinations of rules (“and” or “or” combination of rule membership determinations) may be performed using any T-conorm, as represented by an inverted T symbol or “⊥” such as max(a, b), probabilistic sum of a and b (a+b−a*b), bounded sum, and/or drastic T-conorm; any T-conorm may be used that satisfies the properties of commutativity: ⊥(a, b)=⊥(b, a), monotonicity: ⊥(a, b)≤⊥(c, d) if a≤c and b≤d, associativity: ⊥(a, ⊥(b, c))=⊥(⊥(a, b), c), and identity element of 0. Alternatively or additionally T-conorm may be approximated by sum, as in a “product-sum” inference engine in which T-norm is product and T-conorm is sum. A final output score or other fuzzy inference output may be determined from an output membership function as described above using any suitable defuzzification process, including without limitation Mean of Max defuzzification, Centroid of Area/Center of Gravity defuzzification, Center Average defuzzification, Bisector of Area defuzzification, or the like. Alternatively or additionally, output rules may be replaced with functions according to the Takagi-Sugeno-King (TSK) fuzzy model.

A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an area beneath the first membership function 608 may represent a set of values within first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

First fuzzy set 604 may represent any value or combination of values as described above, including any user data 108 and patient identifiers 120. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 636 that overlaps, first membership function 608 and second membership function 620 may intersect at a point 632 representing a probability, as defined on probability interval, of a match between first fuzzy set 604 and second fuzzy set 616. Alternatively or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, the classification into one or more query categories may indicate a sufficient degree of overlap with fuzzy set representing user data 108 and patient identifiers 120 for combination to occur as described above. Each threshold may be established by one or more user inputs. Alternatively or additionally, each threshold may be tuned by a machine-learning and/or statistical process, for instance and without limitation as described in further detail below.

In an embodiment, a degree of match between fuzzy sets may be used to rank one resource against another. For instance, if both user data 108 and patient identifiers 120 have fuzzy sets, patient identifiers 120 may be generated by having a degree of overlap exceeding a predictive threshold, processor 104 may further rank the two resources by ranking a resource having a higher degree of match more highly than a resource having a lower degree of match. Where multiple fuzzy matches are performed, degrees of match for each respective fuzzy set may be computed and aggregated through, for instance, addition, averaging, or the like, to determine an overall degree of match, which may be used to rank resources; selection between two or more matching resources may be performed by selection of a highest-ranking resource, and/or multiple notifications may be presented to a user in order of ranking.

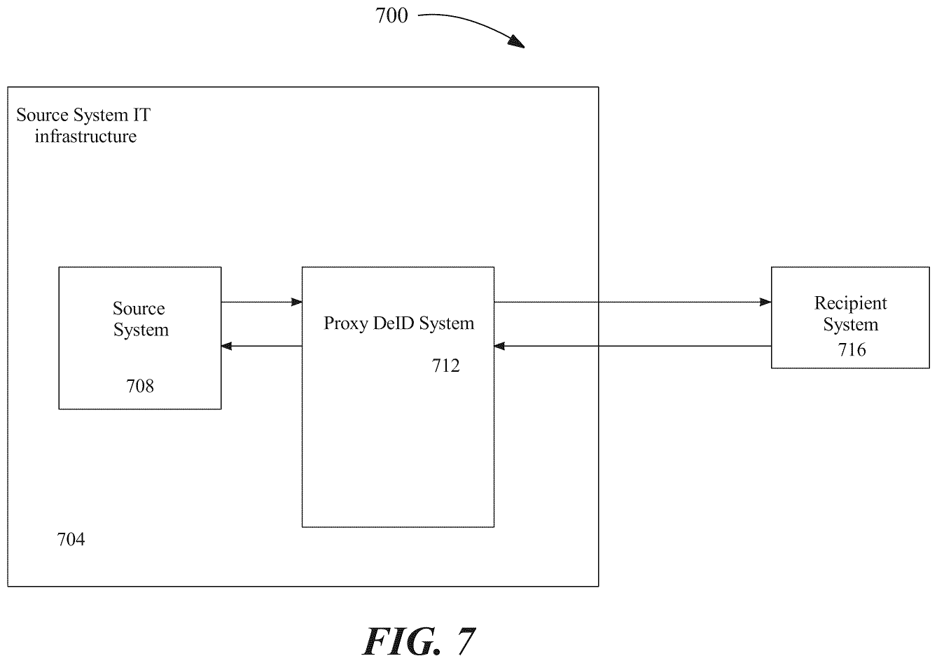

FIG. 7 illustrates an automated one-way tokenization system 700 for sharing medically relevant information in clinical workflows to third parties whilst protecting patient privacy, according to some embodiments. In the illustrated example, the automated one-way tokenization system 700 includes a source system information technology (“IT”) infrastructure 704 and a recipient system 716. The source system IT infrastructure 704 further includes a source system 708 and a proxy deidentification system 712. The automated one-way tokenization system 700 may assign recipient-specific tokenized patient identifiers to any images or text that is shared outside the hospital environment to provide protection from re-identification by triangulation by the multitude of recipients whilst allowing a recipient to provide additional patient information to the hospital using the tokenized identifier.

A lot of clinical workflows involve the use of third parties for various services, e.g., labs for blood work and other measurements, diagnostic imaging centers for various modalities of imaging (CT, X-Ray, MRI etc.), pharmacies, gene sequencing labs to get genetic analysis for samples etc. In these workflows, personally identifiable information (“PIP”) like patient IDs, names, gender, and address are shared. The onus is on recipients to protect the identifiable information.

Additionally, sensitive fields like patient ID, which may be abused to gather more information about a patient, are tokenized (e.g., by use of cryptographic hashes) specifically for each recipient i.e. if the original patient ID of the patient is IDI, in data that is shared with recipient A, all instances of IDI is transformed to the token IDIA; while in data shared with a different recipient, B, all instances of IDI is transformed to IDIB. This ensures that recipients A and B cannot collude to match documents with the same IDs.

In communication back to the source system 708 from the recipient system 716, tokenized information is reconstituted back to their original values and passed into existing workflows within the source system 708. The source system 708 scans responses from the recipient system 716, detects identifier-like elements using the PII detection methodology, and maps any such identifier that matches tokens generated by it back to the original real identifier.

Patients may also be provided communication that was provided to the recipient system 716 for verification purposes to complete workflows (e.g., taking a sample for lab test, dispensing an order etc.)

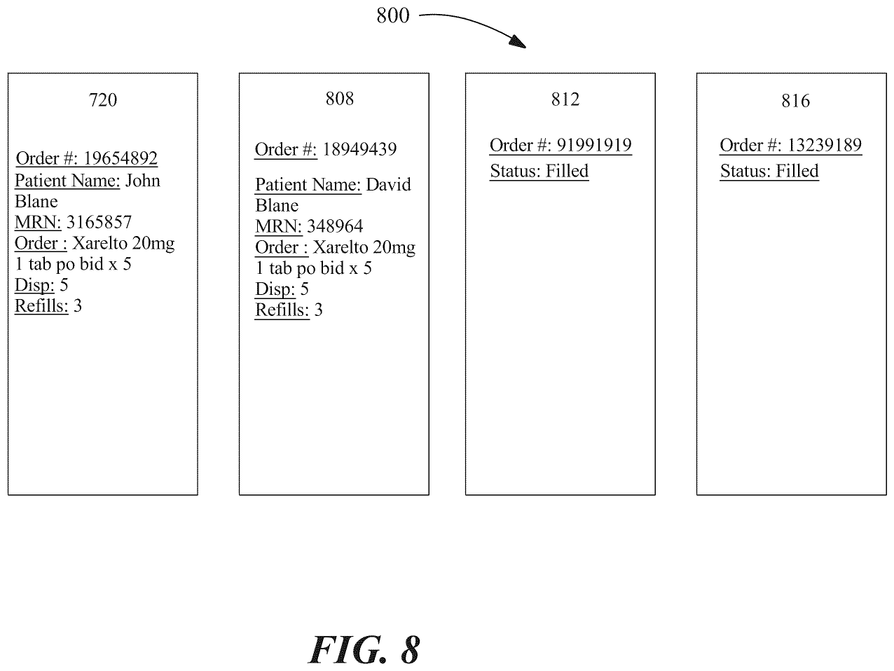

FIG. 8 illustrates a process for generating a medical order using the automated one way tokenization method 800, according to some embodiments. Throughout the illustrated process, four different forms of medical order 804, 808, 812, and 816 are presented.

The first order 804 gets transformed to the second order 804, with its information of order number, MRN, and name obfuscated by the proxy deidentification system 712. In turn, the second order 804 is sent to the recipient system 716. In the second order 804, the actual order details containing drug, dosage, amount (“Disp” for dispense) and number of refills are retained because they are for the recipient system 716 to process.

On completion of processing the order the recipient system 716 sends an acknowledgement to the source system 708 using the tokenized order 808. The proxy deidentification system 712 translates the tokenized order 808 back to the original order 808 and forwards the acknowledgement to the source system 708. The source system 708 continues with the processing as it normally would have in the absence of the proxy.

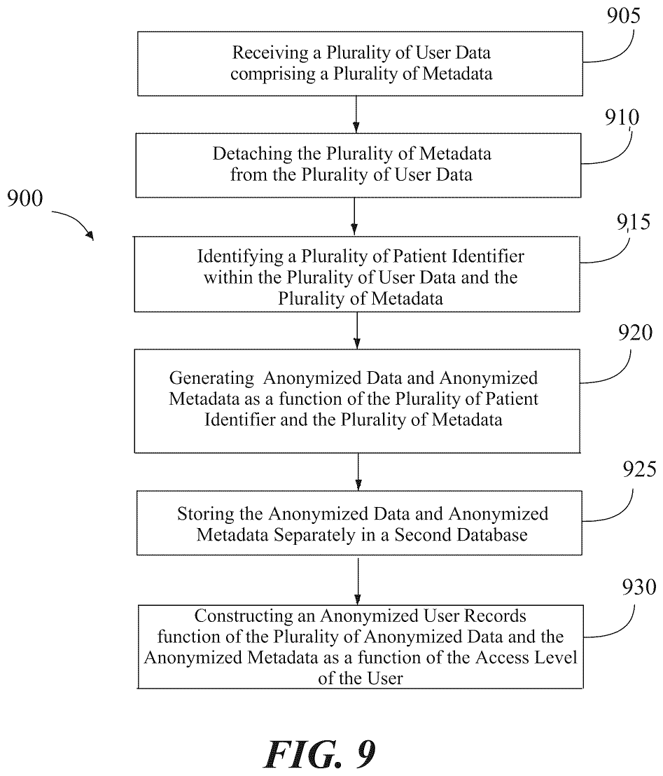

Referring now to FIG. 9, a flow diagram of an exemplary method 900 for anonymizing user data is illustrated. At step 905, method 900 includes receiving, using at least a processor, a plurality of user data comprising a plurality of metadata. This may be implemented as described and with reference to FIGS. 1-8. In an embodiment, the plurality of user data may include a plurality of user imaging data. In an additional embodiment, receiving the plurality of user data may include receiving the plurality of user data from an application program interface.

Still referring to FIG. 9, at step 910, method 900 includes detaching, using the at least a processor, the plurality of metadata form the plurality of user data. This may be implemented as described and with reference to FIGS. 1-8.

Still referring to FIG. 9, at step 915, method 900 includes identifying, using the at least a processor, a plurality of patient identifiers within the plurality of user data and the plurality of metadata. This may be implemented as described and with reference to FIGS. 1-8. In an embodiment, the method further comprises determining, using the at least a processor, an access level of a user as using an authorization identifier; and accessing, using the at least a processor, the plurality of anonymized user records as a function the access level of the user. In some cases, the authorization identifier may include a decentralized token. In an embodiment, identifying the plurality of patient identifiers may include identifying the plurality of patient identifiers within the plurality of user data using named entity recognition system.

Still referring to FIG. 9, at step 920, method 900 includes generating, using the at least a processor, anonymized data and anonymized metadata as a function of the plurality of patient identifiers and the plurality of metadata. This may be implemented as described and with reference to FIGS. 1-8. In an embodiment, generating the plurality of anonymized data of anonymized data may include placing temporal data associated with the plurality of patient identifiers through a temporal shift. In an additional embodiment, generating the plurality of anonymized data comprises aggregating the plurality of patient identifiers. Generating the plurality of anonymized data may include iteratively training an anonymization machine learning model using anonymization training data, wherein anonymization training data comprises a plurality of patient identifiers as inputs correlated to examples of anonymized data as outputs. In an embodiment, anonymizing the plurality of anonymized data may further include identifying at least one gross time identifier and at least one fine grained time identifier in the plurality of patient identifier, obfuscating the at least one gross time identifier, and retaining the at least one fine grained time identifier.

Still referring to FIG. 9, at step 925, method 900 includes storing, using the at least a processor, the anonymized user data, and the anonymized metadata separately in a second database. This may be implemented as described and with reference to FIGS. 1-8.

Still referring to FIG. 9, at step 930, method 900 includes constructing, using at least a processor, an anonymized user record from the anonymized data and the anonymized metadata as a function of an access level of a user. This may be implemented as described and with reference to FIGS. 1-8. In an embodiment, the plurality of anonymized user records may be stored on an immutable sequential listing. In an additional embodiment, method may further include compressing, using the at least a processor, the plurality of anonymized user records as a function of a data compression process.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

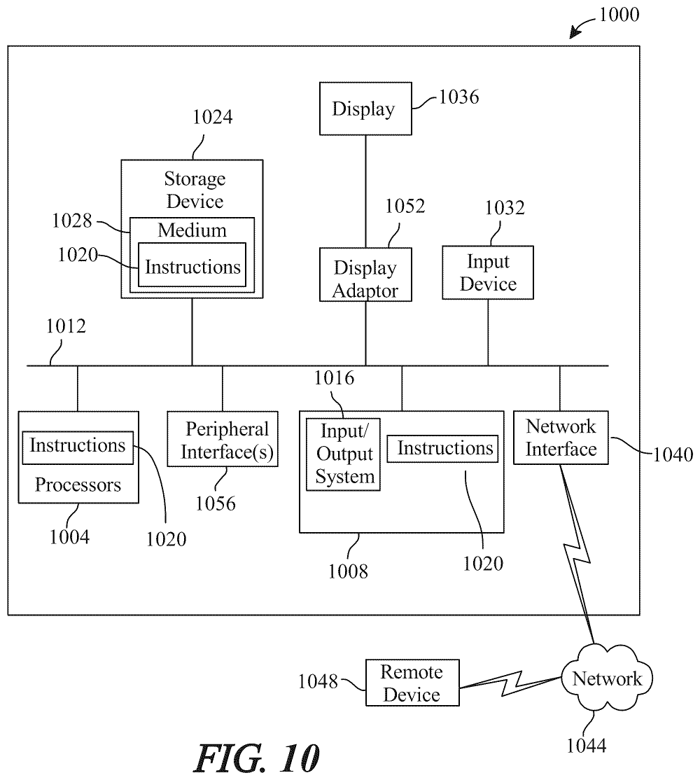

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alphanumeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. An apparatus for anonymizing user data, wherein the apparatus comprises:
at least a processor; and
a memory communicatively connected to the at least a processor, wherein the memory containing instructions configuring the at least a processor to:
receive, from a first database, a plurality of user data comprising a plurality of metadata;
detach the plurality of metadata from the plurality of user data;
identify a plurality of patient identifiers within the plurality of user data and the plurality of metadata;
generate anonymized data and anonymized metadata as a function of the plurality of patient identifiers;
store the anonymized data and the anonymized metadata separately in a second database;
construct an anonymized user record from the anonymized data and the anonymized metadata as a function of an access level of a user.

2. The apparatus of claim 1, wherein the plurality of user data comprises a plurality of user imaging data.

3. The apparatus of claim 1, wherein the memory contains instructions further configuring the processor to determine the access level of a user as using an authorization identifier of the user.

4. The apparatus of claim 1, wherein the generating the plurality of anonymized data comprises:
iteratively training an anonymization machine learning model using anonymization training data, wherein anonymization training data comprises a plurality of patient identifiers as inputs correlated to examples of anonymized data as outputs; and
generating the plurality of anonymized data using the trained anonymization machine learning model.

5. The apparatus of claim 1, wherein the memory contains instructions further configuring the processor to compress the plurality of anonymized user records as a function of a data compression process.

6. The apparatus of claim 1, wherein generating the anonymized data and the anonymized metadata comprises placing temporal data associated with the plurality of patient identifiers through a temporal shift.

7. The apparatus of claim 1, wherein identifying the plurality of patient identifiers comprises identifying the plurality of patient identifiers within the plurality of user data using a named entity recognition system.

8. The apparatus of claim 1, wherein the memory contains instructions further configuring the processor to store, in a sandbox database, the anonymized data and anonymized metadata.

9. The apparatus of claim 8, wherein the memory contains instructions further configuring the processor to store, in the sandbox database, the plurality of user data and the plurality of metadata.

10. The apparatus of claim 1, wherein generating the anonymized data and the anonymized metadata comprises:
identifying at least one gross time identifier and at least one fine grained time identifier in the plurality of patient identifiers;
obfuscating the at least one gross time identifier; and
retaining the at least one fine grained time identifier.

11. A method for anonymizing user data, wherein the method comprises:
receiving, using at least a processor, a plurality of user data comprising a plurality of metadata from a first database;
detaching, using the at least a processor, the plurality of metadata from the plurality of user data;
identifying, using the at least a processor, a plurality of patient identifiers within the plurality of user data and the plurality of metadata;
generating, using the at least a processor, anonymized data and anonymized metadata as a function of the plurality of patient identifiers;
storing, using the at least a processor, the anonymized data and the anonymized metadata separately in a second database;
constructing, using the at least a processor, an anonymized user record from the anonymized data and the anonymized metadata as a function of an access level of a user.

12. The method of claim 11, wherein the plurality of user data comprises a plurality of user imaging data.

13. The method of claim 11, wherein the method further comprises determining, using the at least a processor, the access level of a user as using an authorization identifier of the user.

14. The method of claim 11, wherein the generating the plurality of anonymized data comprises:
iteratively training an anonymization machine learning model using anonymization training data, wherein anonymization training data comprises a plurality of patient identifiers as inputs correlated to examples of anonymized data as outputs; and
generating the plurality of anonymized data using the trained anonymization machine learning model.

15. The method of claim 11, wherein method further comprises compressing, using the at least a processor, the plurality of anonymized user records as a function of a data compression process.

16. The method of claim 11, wherein generating the anonymized data and the anonymized metadata comprises placing temporal data associated with the plurality of patient identifiers through a temporal shift.

17. The method of claim 11, wherein identifying the plurality of patient identifiers comprises identifying the plurality of patient identifiers within the plurality of user data using a named entity recognition system.

18. The method of claim 11, wherein the method further comprises storing, using the at least a processor, the anonymized data and anonymized metadata in a sandbox database.

19. The method of claim 18, wherein the method further comprises storing, using the at least a processor, the plurality of user data and the plurality of metadata in the sandbox database.

20. The method of claim 11, wherein generating the anonymized data and the anonymized metadata comprises:
identifying at least one gross time identifier and at least one fine grained time identifier in the plurality of patient identifiers;
obfuscating the at least one gross time identifier; and
retaining the at least one fine grained time identifier.
